(19) United States
(12) Patent Application Publication
(10) Pub. No.: US 2026/0260750 A1
(43) Pub. Date: Sep. 3, 2026

(54) SYSTEM AND METHOD FOR HIERARCHICAL TUMOR ARTIFICIAL INTELLIGENCE CLASSIFIER TRACES TISSUE OF ORIGIN AND TUMOR TYPE USING DNA METHYLATION

(71) Applicant: The Trustees of Dartmouth College, Hanover, NH (US)

(72) Inventors: Brock C. Christensen, Lebanon, NH (US); Lucas A. Salas, Lebanon, NH (US); Ze Zhang, Hanover, NH (US)

(21) Appl. No.: 19/163,369

(22) PCT Filed: Mar. 8, 2024
(86) PCT No.: PCT/US24/19077

§ 371 Date: Sep. 8, 2025

Related U.S. Application Data
(60) Provisional application No. 63/450,707, filed on Mar. 8, 2023.

(51) Int. Cl. G16H 50/20; G16B 40/00

(52) U.S. Cl. CPC G16H 50/20; G16B 40/00

(57) ABSTRACT

A system and method for operating a novel DNA methylation-based process/algorithm that employs a tumor-type-specific hierarchical model, and thereby, broadens the number of solid tumor types that are traced is provides. This system and method employs multilayer perceptron models in combination with the discriminatory CpGs specific to tumor type in each layer in the hierarchy. The tracing cancerous tissue uses a processor and a user interface operating data input process receives information related to the patient's cancerous tissue based on DNA methylation. A data store defines layers of information arranged in a hierarchy relative to types of cancerous conditions in tissue based on DNA methylation therein. An analysis process, having a trained machine learning process, compares DNA methylation characteristics in the patient's tissue to the layers, and performs a match so as to trace the cancerous condition. A diagnostic process provides a user with information relative to the match.

### RELATED APPLICATION

This application claims the benefit of co-pending U.S. Patent Application Ser. No. 63/450,707, entitled SYSTEM AND METHOD FOR HIERARCHICAL TUMOR ARTIFICIAL INTELLIGENCE CLASSIFIER TRACES TISSUE OF ORIGIN AND TUMOR TYPE USING DNA METHYLATION, filed Mar. 8, 2023, the teachings of which are expressly incorporated herein by reference.

### STATEMENT REGARDING FEDERALLY SPONSORED RESEARCH OR DEVELOPMENT

This invention was made with U.S. government support under Grant Numbers W81XWH-20-1-0778 awarded by the U.S. Congressionally Directed Medical Research Programs (CDMRP)/Department of Defense (DOD), P20GM104416/8299 awarded by the U.S. National Institute of General Medical Sciences (NIGMS) and R01 CA253976, awarded by the National Institutes of Health (NIH)/National Cancer Institute (NCI). The government has certain rights in this invention.

### FIELD OF THE INVENTION

This invention relates to systems and methods for diagnosis of cancerous conditions from cellular samples based upon deconvolution of DNA methylation data.

### BACKGROUND OF THE INVENTION

Cancer is the second leading cause of death in the United States, following heart disease. One thousand six hundred seventy deaths are projected to be caused by cancer per day in 2023, aggregating to an estimated 609,820 cancer deaths in the year. Cancer metastasis is the primary cause of cancer mortality, representing around 90% of cancer deaths. Metastatic cancer hallmarks a significantly worse prognosis with limited treatment options and a low response rate. Cancer metastasis occurs when advanced tumor cells acquire the ability to detach from the primary tumor tissue, migrate through the blood and lymphatic vessels, invade a distal tissue site, and proliferate at the new site. Although primary tumor site for metastatic cancer can usually be recognized by clinical procedures like imaging, immunohistochemistry (IHC) tests, and pathological analyses, cancer of unknown primary (CUP) persists for advanced cancer with high tumor cell heterogeneity, atypical morphological patterns, and absence of identifiable features. CUP is defined as metastatic cancer for which the primary anatomic origin cannot be identified. CUP accounts for 2-5% of all cancers and marks significantly worse clinical outcomes. The median survival rate is less than one year for CUP patients. Therapeutic strategies today are highly dependent on the clinical, pathological, and molecular profiles of cancer See Varadhachary, G. R. and Raber, M. N. (2014) Cancer of unknown primary site. N Engl J Med, 371, 757-765, and Rassy, E. and Pavlidis, N. (2020) Progress in refining the clinical management of cancer of unknown primary in the molecular era. Nat Rev Clin Oncol, 17, 541-554. CUP poses the challenge of proper and timely treatment for patients, resulting in unfavorable clinical outcomes. Although clinical tools, e.g. IHC, pathology test, are available for identifying CUP, only 25% of CUP can be diagnosed due to the limited sensitivity and specificity of the traditional methods.

DNA methylation is an epigenetic modification that regulates gene expression and is essential to establishing and preserving cellular identity. See Bogdanovic, O. and Lister, R. (2017) DNA methylation and the preservation of cell identity. Curr Opin Genet Dev, 46, 9-14. See Salas, L. A., Zhang, Z., Koestler, D. C., Butler, R. A., Hansen, H. M., Molinaro, A. M., Wiencke, J. K., Kelsey, K. T. and Christensen, B. C. (2022) Enhanced cell deconvolution of peripheral blood using DNA methylation for high-resolution immune profiling. Nat Commun, 13, 761; Arneson, D., Yang, X. and Wang, K. (2020); MethylResolver—a method for deconvoluting bulk DNA methylation profiles into known and unknown cell contents. Commun Biol, 3, 422; Chakravarthy, A., Furness, A., Joshi, K., Ghorani, E., Ford, K., Ward, M. J., King, E. V., Lechner, M., Marafioti, T., Quezada, S. A. et al. (2018) Pan-cancer deconvolution of tumour composition using DNA methylation. Nat Commun, 9, 3220; and Zhang, Z., Wiencke, J. K., Kelsey, K. T., Koestler, D. C., Christensen, B. C. and Salas, L. A. (2022) HiTIMED: hierarchical tumor immune microenvironment epigenetic deconvolution for accurate cell type resolution in the tumor microenvironment using tumor-type-specific DNA methylation data. J Transl Med, 20, 516. In recent years, DNA methylation has been widely utilized as a biomarker for cell typing in blood and the tumor microenvironment. Furthermore, cell-free DNA methylation profiling is beginning to show promise for early detection and classification of cancer. See Chen, X., Gole, J., Gore, A., He, Q., Lu, M., Min, J., Yuan, Z., Yang, X., Jiang, Y., Zhang, T. et al. (2020) Non-invasive early detection of cancer four years before conventional diagnosis using a blood test. Nat Commun, 11, 3475, and Li, W. and Zhou, X. J. (2020) Methylation extends the reach of liquid biopsy in cancer detection. Nat Rev Clin Oncol, 17, 655-656. As a well-established biomarker for cell identity, DNA methylation holds promising value for distinguishing heterogeneous tumor subtypes, especially for CUP. Genome-wide DNA methylation arrays provide a standardized and cost-effective approach to measuring DNA methylation. See Schumacher, A., Kapranov, P., Kaminsky, Z., Flanagan, J., Assadzadeh, A., Yau, P., Virtanen, C., Winegarden, N., Cheng, J., Gingeras, T. et al. (2006) Microarray-based DNA methylation profiling: technology and applications. Nucleic Acids Res, 34, 528-542. The high-dimensional methylation data in combination with artificial intelligence (AI) technologies promises new opportunities to efficiently trace tumor tissue of origin that may have clinical significance, especially for metastasized cancer and CUP.

The advance of AI in biomedical science enables translational technology from sophisticated computational tasks and high-dimensional data to potential clinical usage. See Noorbakhsh-Sabet, N., Zand, R., Zhang, Y. and Abedi, V. (2019) Artificial Intelligence Transforms the Future of Health Care. Am J Med, 132, 795-801. AI-powered medicine provides streamlined analysis and efficient processing of complex clinical and biomedical data, especially in pathology and laboratory medicine. See Cui, M. and Zhang, D. Y. (2021) Artificial intelligence and computational pathology. Lab Invest, 101, 412-422. In the past decade, by virtue of the progress made in computational capacity and new technologies for genomic sequencing, publicly available biomedical data repertoires are established and structured to serve the scientific community with easily accessible data sets, e.g. The Cancer Genome Atlas (TCGA), Gene Expression Omnibus (GEO), and ArrayExpress. Combined with advances in AI, researchers have utilized the enormity of publicly available data (accessed over various communication/data networks, such as the Internet) to study genomic biology. One of the most popular examples of these technologies is the AI-powered protein folding prediction research. See Jumper, J., Evans, R., Pritzel, A., Green, T., Figurnov, M., Ronneberger, O., Tunyasuvunakool, K., Bates, R., Zidek, A., Potapenko, A. et al. (2021) Highly accurate protein structure prediction with AlphaFold. Nature, 596, 583-589. At the genomic level, studies are beginning to show the application of machine learning modeling to integrate multiomics information for disease diagnosis and prognostication, which is especially relevant for studying cancer biology. See Huang, S., Cai, N., Pacheco, P. P., Narrandes, S., Wang, Y. and Xu, W. (2018) Applications of Support Vector Machine (SVM) Learning in Cancer Genomics. Cancer Genomics Proteomics, 15, 41-51; Poirion, O. B., Jing, Z., Chaudhary, K., Huang, S. and Garmire, L. X. (2021) DeepProg: an ensemble of deep-learning and machine-learning models for prognosis prediction using multi-omics data. Genome Med, 13, 112; and Wang, T., Shao, W., Huang, Z., Tang, H., Zhang, J., Ding, Z. and Huang, K. (2021) MOGONET integrates multi-omics data using graph convolutional networks allowing patient classification and biomarker identification. Nat Commun, 12, 3445.

In recent years, researchers have demonstrated high performance of DNA methylation-based machine learning models tracing the tissue origin of tumor cells. See Zheng, C. and Xu, R. (2020) Predicting cancer origins with a DNA methylation-based deep neural network model. PLoS One, 15, e0226461; Modhukur, V., Sharma, S., Mondal, M., Lawarde, A., Kask, K., Sharma, R. and Salumets, A. (2021) Machine Learning Approaches to Classify Primary and Metastatic Cancers Using Tissue of Origin-Based DNA Methylation Profiles. Cancers (Basel), 13; and Moran, S., Martinez-Cardus, A., Sayols, S., Musulen, E., Balana, C., Estival-Gonzalez, A., Moutinho, C., Heyn, H., Diaz-Lagares, A., de Moura, M. C. et al. (2016) Epigenetic profiling to classify cancer of unknown primary: a multicentre, retrospective analysis. Lancet Oncol, 17, 1386-1395. However, previous works have been limited by the number and variation of cancer types and validation data sets considered, especially for metastasized cancers and circulating cell-free DNA from cancer patients. Furthermore, previous models have been devised based on tissue site instead of tumor type, generating potential problems of indistinguishable tumor subtypes from the same site, e.g., esophageal squamous cell carcinoma versus esophageal adenocarcinoma. Although there is some work to illustrate the utility and replicability of the DNA methylation-based machine learning models on tracing tissue of origin for cancers, these works lacked tumor-type specificity and have not been made readily accessible and user-friendly to the general scientific community. A previous study used a hierarchical modeling approach to address the challenge of deconvolving cell types that are of same lineage in the tumor microenvironment. See Zhang, above, and refer also to commonly assigned PCT Application Serial No. PCT/US23/12438, SYSTEM AND METHOD FOR HIERARCHICAL TUMOR IMMUNE MICROENVIRONMENT EPIGENETIC DECONVOLUTION, filed Feb. 6, 2022, the teachings of which are incorporated herein by reference.

It is desirable to provide a technique for tracing tumor characteristics and origins that is straightforward to use and radially available to practitioners and other interested parties.

### SUMMARY OF THE INVENTION

To address the limitations of existing methods and enhance the accuracy, utility and accessibility of tumor tracing, this invention provides a system and method for operating a novel DNA methylation-based process/algorithm that employs a tumor-type-specific hierarchical model, and thereby, broadens the number of solid tumor types that are traced. This system and method can be termed Hierarchical Tumor Artificial Intelligence Classifier (HiTAIC), and employs multilayer perceptron models in combination with the discriminatory CpGs specific to tumor type in each layer in the hierarchy, to trace tumor tissue of origin and subtypes in (e.g.) 27 primary and metastasized cancers. HiTAIC's ability to trace tumor tissue of origin with high resolution provides valuable application to clinical CUP identification.

In an illustrative embodiment, a system and method for tracing cancerous tissue in a patient using a processor and a user interface is provided. A data input process receives information related to the patient's cancerous tissue based on DNA methylation. A data store defines a plurality of layers of information arranged in a hierarchy relative to types of cancerous conditions in tissue based on DNA methylation therein. An analysis process includes a trained machine learning process, which compares DNA methylation characteristics in the patient's tissue to the layers of information, and performs a match so as to trace the cancerous condition. A diagnostic process provides a user with information relative to the match. Illustratively, the first layer of the plurality of layers is selected from a group of conditions consisting of: Adenocarcinoma, Glioma, Melanoma, Mesothelioma, Pheochromocytoma and Paraganglioma, Sarcoma, Squamous cell carcinoma, Testicular germ cell tumor, and Thymoma. The first layer of the plurality of layers, relative to Adencarcinoma, can include a second layer selected from a group of cancerous conditions consisting of those related to: Adrenocortical, Bladder, Breast, Cervical, Colorectal, Endometrial, Kidney chromophobe, Kidney clear cell, Kidney papillary cell, Liver hepatocellular, Esophageal and stomach, Lung, Ovarian, Pancreatic, Prostate, and Thyroid. The first layer, relative to Squamous cell carcinoma, can include a second layer selected from a group of cancerous conditions consisting of those related to: Cervical, Esophageal and head and neck, and Lung. The first layer, relative to Melanoma, includes a second layer selected from a group on cancerous conditions consisting of those related to: Eye uveal and Skin cutaneous. The analysis process can be arranged to trace cancerous conditions from DNA methylation information in metastasized cancerous tissue. The data store can include publicly available information accessed through a public data communication network, such as the Internet. The machine learning process can be trained using classifiers related to predetermined DNA methylation characteristics for related cancerous conditions. Illustratively, at least one of the data input process, the analysis process and the diagnostic process is operated using a processor on a user-controlled computing device with a user interface. Generally, the system and method can be employed for diagnosing and reporting upon cancerous conditions to a user.

### BRIEF DESCRIPTION OF THE DRAWINGS

The invention description below refers to the accompanying drawings, of which:

FIG. 1 is a diagram showing a Hierarchical Tumor Artificial Intelligence Classifier (HiTAIC) tumor classifier hierarchy with two layers for twenty-seven cancer types;

FIG. 2 is a flow diagram showing HiTAIC model building, validation, and application;

FIGS. 3A-3D are heatmaps showing DNA methylation states of cancer discriminating CpGs for Layer 1 major cancer types (FIG. 3A), Layer 2A adenocarcinoma subtypes (FIG. 3B), Layer 2B squamous cell carcinoma subtypes (FIG. 3C), and Layer 2C melanoma subtypes (FIG. 3D);

FIG. 3E is a graph showing the number of overlapping CpGs across four libraries employed by the HiTAIC for the system and method herein;

FIGS. 4A-4D are graphs showing GREAT Gene Ontology pathways enriched for CpGs discerning major cancer types (FIG. 4A), adenocarcinoma subtypes (FIG. 4B), squamous cell carcinoma subtypes (FIG. 4C), and melanoma subtypes (FIG. 4D);

FIGS. 4E-4H are tables showing genomic context enriched for Layer 1, Layer 2A, Layer 2B, and Layer 2C cancer discerning CpGs employed by HiTAIC for the system and method herein;

FIG. 5 is a block diagram showing a generalized computing environment for performing the processes and steps of the system and method herein; and

FIG. 6 is a table showing the HiTAIC architecture for the system and method herein, according to an exemplary embodiment.

### DETAILED DESCRIPTION

### I. Abbreviations

To assist the reader, the following abbreviations are used in the Specification and Drawings herein relative to the terms listed as follows:
AI: artificial intelligence;
BLCA: bladder urothelial carcinoma;
BRCA: breast invasive carcinoma;
CESC: cervical squamous cell carcinoma and endocervical adenocarcinoma;
CORE: colorectal adenocarcinoma;
CUP: cancer of unknown origin;
ESCA: esophageal carcinoma;
GBM: glioblastoma multiforme;
GEO: gene expression omnibus;
HNSC: head and neck squamous cell carcinoma;
IHC: immunohistochemistry;
KICH: kidney chromophobe carcinoma;
KIRC: kidney renal clear cell carcinoma;
KIRP: kidney renal papillary cell carcinoma;
LIHC: liver hepatocellular carcinoma;
LUAD: lung adenocarcinoma;
LUSC: lung squamous cell carcinoma;
PAAD: pancreatic adenocarcinoma;
PRAD: prostate adenocarcinoma;
SKCM: cutaneous melanoma;
STAD: stomach adenocarcinoma;
TCGA: The Cancer Genome Atlas;
THCA: thyroid carcinoma; and
UCEC: uterine corpus endometrial carcinoma

### II. Overview Methods, Materials and Technical Considerations

### A. Discovery Dataset and Quality Control

By way of background, a study has been undertaken for the initial discovery data sets included DNA methylation microarray data from 7932 samples across 30 cancer types with tagged known primary from TCGA, which is a publicly available cancer data repertoire. 194 leukemia samples are removed as only solid tumors have been targeted. 99 ovarian tumor samples are added to the discovery data set from GEO data set GSE133556 due to limited ovarian tumor sample size on TCGA. 102 samples are excluded from the discovery data set because of the ambiguity of the tumor subtypes. The ambiguous tumors are tumor subtypes that typically do not fall into any category of the Hierarchical Tumor Artificial Intelligence Classifier (HiTAIC) hierarchy at present (but may do so with further development in a manner clear to those of skill).

Table 1, summarizes the discovery data set based on cancer site, tumor subtype, and exclusion criteria. In total, 7735 tumor samples from 27 cancer types are included in the discovery data set (Table 1A, below). The discovery data set is then randomly split into 80% training and 20% testing for model training and testing. The methylation data quality control process retains CpGs that measured on both Illumina HumanMethylation450k and HumanMethylationEPIC platforms to accommodate cross-platform applications. The SeSAMe (version 1.8.2) pipeline from Bioconductor is used to preprocess the data, including data normalization and quality control. See Zhou, W., Triche, T. J., Jr., Laird, P. W. and Shen, H. (2018) SeSAMe: reducing artifactual detection of DNA methylation by Infinium BeadChips in genomic deletions. Nucleic Acids Res, 46, e123. Cross-reactive probes, single-nucleotide polymorphism (SNP)-related probes, sex chromosome probes, non-CpG probes, and low-quality probes (pOOBHA>0.05) are masked in the analysis. 384,640 CpGs are retained after this process.

By way of further background and understanding, it is noted that CpG sites (also termed simply, “CpGs” herein) are regions of DNA in which a cytosine nucleotide is followed by a guanine nucleotide in a linear sequence of bases along its 5′(prime)→3′(prime) direction. CpG sites generally reside in genomic regions called CpG islands. Notably, cytosines in CpG dinucleotides can be methylated using known processes to derive 5-methylcytosines.

TABLE 1
Discovery data set based on cancer site, tumor subtype, and exclusion criteria.

| | | | | | Keep |
|---|---|---|---|---|---|
| | | TCGA | | | (1: Yes; |
| Primary Diagnosis | HiTAIC Class | Type | N | Source | 0: No) |
| Abdominal fibromatosis | Sarcoma | SARC | 1 | TCGA | 1 |
| Acinar cell carcinoma | Adenocarcinoma | LUAD | 22 | TCGA | 1 |
| Acral lentiginous melanoma, malignant | Melanoma | SKCM | 2 | TCGA | 1 |
| Acute myeloid leukemia, NOS | NA | LAML | 194 | TCGA | 0 |
| Adenocarcinoma in tubolovillous adenoma | Adenocarcinoma | READ | 8 | TCGA | 1 |
| Adenocarcinoma with mixed subtypes | Adenocarcinoma | LUAD | 93 | TCGA | 1 |
| Adenocarcinoma with mixed subtypes | Adenocarcinoma | PAAD | 1 | TCGA | 1 |
| Adenocarcinoma with mixed subtypes | Adenocarcinoma | PRAD | 3 | TCGA | 1 |
| Adenocarcinoma with mixed subtypes | Adenocarcinoma | READ | 2 | TCGA | 1 |
| Adenocarcinoma, endocervical type | Adenocarcinoma | CESC | 21 | TCGA | 1 |
| Adenocarcinoma, intestinal type | Adenocarcinoma | STAD | 77 | TCGA | 1 |
| Adenocarcinoma, NOS | Adenocarcinoma | CESC | 7 | TCGA | 1 |
| Adenocarcinoma, NOS | Adenocarcinoma | COAD | 242 | TCGA | 1 |
| Adenocarcinoma, NOS | Adenocarcinoma | ESCA | 87 | TCGA | 1 |
| Adenocarcinoma, NOS | Adenocarcinoma | LUAD | 272 | TCGA | 1 |
| Adenocarcinoma, NOS | Adenocarcinoma | PAAD | 20 | TCGA | 1 |
| Adenocarcinoma, NOS | Adenocarcinoma | PRAD | 485 | TCGA | 1 |
| Adenocarcinoma, NOS | Adenocarcinoma | READ | 76 | TCGA | 1 |
| Adenocarcinoma, NOS | Adenocarcinoma | STAD | 137 | TCGA | 1 |
| Adenocarcinoma, NOS | Adenocarcinoma | UCEC | 1 | TCGA | 1 |
| Adenoid cystic carcinoma | Adenocarcinoma | BRCA | 1 | TCGA | 1 |
| Adenosquamous carcinoma | Squamous cell carcinoma | CESC | 5 | TCGA | 1 |
| Adrenal cortical carcinoma | Adenocarcinoma | ACC | 80 | TCGA | 1 |
| Aggressive fibromatosis | Sarcoma | SARC | 1 | TCGA | 1 |
| Amelandtic melanoma | Melanoma | SKCM | 3 | TCGA | 1 |
| Astrocytoma, anaplastic | NA | LGG | 130 | TCGA | 0 |
| Astrocytoma, NOS | NA | LGG | 64 | TCGA | 0 |
| Basal cell carcinoma, NOS | NA | BRCA | 1 | TCGA | 0 |
| Basaloid squamous cell carcinoma | Squamous cell carcinoma | CESC | 1 | TCGA | 1 |
| Basaloid squamous cell carcinoma | Squamous cell carcinoma | ESCA | 1 | TCGA | 1 |
| Basaloid squamous cell carcinoma | Squamous cell carcinoma | HNSC | 10 | TCGA | 1 |
| Basaloid squamous cell carcinoma | Squamous cell carcinoma | LUSC | 11 | TCGA | 1 |
| Bronchio-alveolar carcinoma, mucinous | Adenocarcinoma | LUAD | 5 | TCGA | 1 |
| Bronchiolo-alveolar adenocarcinoma, NOS | Adenocarcinoma | LUAD | 3 | TCGA | 1 |
| Bronchiolo-alveolar carcinoma, non- | Adenocarcinoma | LUAD | 19 | TCGA | 1 |
| mucinous | | | | | |
| Carcinoma, diffuse type | Adenocarcinoma | STAD | 64 | TCGA | 1 |
| Carcinoma, NOS | NA | BLCA | 1 | TCGA | 0 |
| Carcinoma, NOS | NA | BRCA | 1 | TCGA | 0 |
| Carcinoma, NOS | NA | THCA | 1 | TCGA | 0 |
| Carcinoma, undifferentiated, NOS | NA | PAAD | 1 | TCGA | 0 |
| Carcinoma, undifferentiated, NOS | NA | UCEC | 1 | TCGA | 0 |
| Carcinosarcoma, NOS | NA | UCS | 11 | TCGA | 0 |
| Clear cell adenocarcinoma, NOS | Adenocarcinoma | KIRC | 305 | TCGA | 1 |
| Clear cell adenocarcinoma, NOS | Adenocarcinoma | LIHC | 1 | TCGA | 1 |
| Clear cell adenocarcinoma, NOS | Adenocarcinoma | LUAD | 1 | TCGA | 1 |
| Combined hepatocellular carcinoma and | Adenocarcinoma | LIHC | 7 | TCGA | 1 |
| cholangiocarcinoma | | | | | |
| Cystadenocarcinoma, NOS | Adenocarcinoma | OV | 1 | TCGA | 1 |
| Dedifferentiated liposarcoma | Sarcoma | SARC | 58 | TCGA | 1 |
| Embryonal carcinoma, NOS | Testicular germ cell tumor | TGCT | 26 | TCGA | 1 |
| Endometrioid adenocarcinoma, NOS | Adenocarcinoma | CESC | 3 | TCGA | 1 |
| Endometrioid adenocarcinoma, NOS | Adenocarcinoma | UCFC | 305 | TCGA | 1 |
| Endometrioid adenocarcinoma, secretory | Adenocarcinoma | UCEC | 2 | TCGA | 1 |
| variant | | | | | |
| Epithelioid cell melanoma | Melanoma | SKCM | 7 | TCGA | 1 |
| Epithelioid cell melanoma | Melanoma | UVM | 12 | TCGA | 1 |
| Epithelioid mesothelioma, malignant | Mesothelioma | MESO | 58 | TCGA | 1 |
| Extra-adrenal paraganglioma, malignant | Pheochromocytoma and | PCPG | 6 | TCGA | 1 |
| | Paraganglioma | | | | |
| Extra-adrenal paraganglioma, NOS | Pheochromocytoma and | PCPG | 18 | TCGA | 1 |
| | Paraganglioma | | | | |
| Fibromyxosarcoma | Sarcoma | SARC | 25 | TCGA | 1 |
| Fibrous mesothelioma, malignant | Mesothelioma | MESO | 1 | TCGA | 1 |
| Follicular adenocarcinoma, NOS | Adenocarcinoma | THCA | 1 | TCGA | 1 |
| Follicular carcinoma, minimally invasive | Adenocarcinoma | THCA | 1 | TCGA | 1 |
| Giant cell sarcoma | Sarcoma | SARC | 3 | TCGA | 1 |
| Glioblastoma | Glioma | GBM | 138 | TCGA | 1 |
| Hepatocellular carcinoma, clear cell type | Adenocarcinoma | LIHC | 4 | TCGA | 1 |
| Hepatocellular carcinoma, fibrolamellar | Adenocarcinoma | LIHC | 3 | TCGA | 1 |
| Hepatocellular carcinoma, NOS | Adenocarcinoma | LIHC | 361 | TCGA | 1 |
| Hepatocellular carcinoma, spindle cell | Adenocarcinoma | LIHC | 1 | TCGA | 1 |
| variant | | | | | |
| Infiltrating duct and lobular carcinoma | Adenocarcinoma | BRCA | 24 | TCGA | 1 |
| Infiltrating duct carcinoma, NOS | Adenocarcinoma | BRCA | 507 | TCGA | 1 |
| Infiltrating duct carcinoma, NOS | Adenocarcinoma | PAAD | 149 | TCGA | 1 |
| Infiltrating duct carcinoma, NOS | Adenocarcinoma | PRAD | 9 | TCGA | 1 |
| Infiltrating duct mixed with other types of | Adenocarcinoma | BRCA | 10 | TCGA | 1 |
| carcinoma | | | | | |
| Infiltrating lobular mixed with other types | Adenocarcinoma | BRCA | 7 | TCGA | 1 |
| of carcinoma | | | | | |
| Intraductal micropapillary carcinoma | Adenocarcinoma | BRCA | 4 | TCGA | 1 |
| Intraductal papillary adenocarcinoma with | Adenocarcinoma | BRCA | 4 | TCGA | 1 |
| invasion | | | | | |
| Large cell neuroendocrine carcinoma | NA | BRCA | 1 | TCGA | 0 |
| Leiomyosarcoma, NOS | Sarcoma | SARC | 102 | TCGA | 1 |
| Lentigo maligna melanoma | Melanoma | SKOM | 1 | TCGA | 1 |
| Liposarcoma, well differentiated | Sarcoma | SARC | 1 | TCGA | 1 |
| Lobular carcinoma, NOS | Adenocarcinoma | BRCA | 178 | TCGA | 1 |
| Malignant lymphoma, large B-cell, diffuse, | NA | DLBC | 48 | TCGA | 0 |
| NOS | | | | | |
| Malignant melanoma, NOS | Melanoma | SKCM | 69 | TCGA | 1 |
| Malignant melanoma, NOS | Melanoma | UVM | 1 | TCGA | 1 |
| Malignant peripheral nerve sheath tumor | Sarcoma | SARC | 9 | TCGA | 1 |
| Medullary carcinoma, NOS | NA | BRCA | 6 | TCGA | 0 |
| Mesodermal mixed tumor | NA | UCS | 1 | TCGA | 0 |
| Mesothelioma, biphasic, malignant | Mesothelioma | MESO | 22 | TCGA | 1 |
| Mesothelioma, malignant | Mesothelioma | MESO | 6 | TCGA | 1 |
| Metaplastic carcinoma, NOS | NA | BRCA | 13 | TCGA | 0 |
| Micropapillary carcinoma, NOS | Adenocarcinoma | LUAD | 2 | TCGA | 1 |
| Mixed epitheliold and spindle cell | Melanoma | SKCM | 1 | TCGA | 1 |
| melanoma | | | | | |
| Mixed epithelioid and spindle cel | Melanoma | UVM | 39 | TCGA | 1 |
| melanoma | | | | | |
| Mixed germ cell tumor | Testicular germ cell tumor | TGCT | 28 | TCGA | 1 |
| Mixed glioma | NA | LGG | 131 | TCGA | 0 |
| Mucinous adenocarcinoma | Adenocarcinoma | BRCA | 15 | TCGA | 1 |
| Mucinous adenocarcinoma | Adenocarcinoma | COAD | 38 | TCGA | 1 |
| Mucinous adenocarcinoma | Adenocarcinoma | ESCA | 1 | TCGA | 1 |
| Mucinous adenocarcinoma | Adenocarcinoma | LUAD | 13 | TCGA | 1 |
| Mucinous adenocarcinoma | Adenocarcinoma | PAAD | 5 | TCGA | 1 |
| Mucinous adenocarcinoma | Adenocarcinoma | PRAD | 1 | TCGA | 1 |
| Mucinous adenocarcinoma | Adenocarcinoma | READ | 6 | TCGA | 1 |
| Mucinous adenocarcinoma | Adenocarcinoma | STAD | 20 | TCGA | 1 |
| Mucinous adenocarcinoma, endocervical | Adenocarcinoma | CESC | 17 | TCGA | 1 |
| type | | | | | |
| Mullerian mixed tumor | NA | UCS | 45 | TCGA | 0 |
| Myxoid leiomyosarcoma | Sarcoma | SARC | 3 | TCGA | 1 |
| Neuroendocrine carcinoma, NOS | NA | PAAD | 8 | TCGA | 0 |
| Nodular melanoma | Melanoma | SKCM | 17 | TCGA | 1 |
| Nonencapsulated sclerosing carcinoma | NA | THCA | 4 | TCGA | 0 |
| Oligodendroglioma, anaplastic | NA | LGG | 78 | TCGA | 0 |
| Oligodendroglioma, NOS | NA | LGG | 112 | TCGA | 0 |
| Oxyphilic adenocarcinoma | Adenocarcinoma | THCA | 1 | TCGA | 1 |
| Paget disease and infiltrating duct | Adenocarcinoma | BBCA | 2 | TCGA | 1 |
| carcinoma of breast | | | | | |
| Papillary adenocarcinoma, NOS | Adenocarcinoma | BLCA | 1 | TCGA | 1 |
| Papillary adenocarcinoma, NOS | Adenocarcinoma | COAD | 2 | TCGA | 1 |
| Papillary adenocarcinoma, NOS | Adenocarcinoma | KIRP | 275 | TCGA | 1 |
| Papillary adenocarcinoma, NOS | Adenocarcinoma | LUAD | 21 | TCGA | 1 |
| Papillary adenocarcinoma, NOS | Adenocarcinoma | STAD | 8 | TCGA | 1 |
| Papillary adenocarcinoma, NOS | Adenocarcinoma | THCA | 356 | TCGA | 1 |
| Papillary carcinoma, columnar cell | Adenocarcinoma | THCA | 38 | TCGA | 1 |
| Papillary carcinoma, follicular variant | Adenocarcinoma | THCA | 105 | TCGA | 1 |
| Papillary carcinoma, NOS | Adenocarcinoma | BRCA | 2 | ICGA | 1 |
| Papillary serous cystadenocarcinoma | Adenocarcinoma | OV | 4 | TCGA | 1 |
| Papillary serous cystadenocarcinoma | Adenocarcinoma | UCEC | 4 | TCGA | 1 |
| Papillary squamous cell carcinoma | Squamous cell carcinoma | CESC | 1 | TCGA | 1 |
| Papillary squamous cell carcinoma | Squamous cell carcinoma | LUSC | 4 | TCGA | 1 |
| Papillary transitional cell carcinoma | Adenocarcinoma | BLCA | 66 | TCGA | 1 |
| Paraganglioma, malignant | Pheochromocytoma and | PCPG | 2 | TCGA | 1 |
| | Paraganglioma | | | | |
| Paraganglioma, NOS | Pheochromocytoma and | PCPG | 4 | TCGA | 1 |
| | Paraganglioma | | | | |
| Pheochromocytoma, malignant | Pheochromocytoma and | PCPG | 39 | TCGA | |
| | Paraganglioma | | | | 1 |
| Pheochromocytoma, NOS | Pheochromocytoma and | PCPG | 110 | TCGA | 1 |
| | Paraganglioma | | | | |
| Phyllodes tumor, malignant | Adenocarcinoma | BRCA | 2 | TCGA | 1 |
| Pleomorphic carcinoma | Adenocarcinoma | BRCA | 3 | TCGA | 1 |
| Pleomorphic liposarcoma | Sarcoma | SARC | 2 | TCGA | 1 |
| Renal cell carcinoma, chromophobe type | Adenocarcinoma | KICH | 66 | TCGA | 1 |
| Renal cell carcinoma, NOS | Adenocarcinoma | KIRC | 14 | TCGA | 1 |
| Secretory carcinoma of breast | Adenocarcinoma | BRCA | 1 | TCGA | 1 |
| Seminoma, NOS | Testicular germ cell tumor | TGCT | 66 | TCGA | 1 |
| Serous cystadenocarcinoma, NOS | Adenocarcinoma | OV | 5 | TCGA | 1 |
| Serous cystadenocarcinoma, NOS | Adenocarcinoma | UCEC | 117 | TCGA | 1 |
| Serous surface papillary carcinoma | Adenocarcinoma | UCEC | 1 | TCGA | 1 |
| Signet ring cell carcinoma | Adenocarcinoma | LUAD | 1 | TCGA | 1 |
| Signet ring cell carcinoma | Adenocarcinoma | STAD | 14 | TCGA | 1 |
| Solid carcinoma, NOS | Adenocarcinoma | LUAD | 6 | TCGA | 1 |
| Spindle cell melanoma, NOS | Melanoms | SKOM | 2 | TCGA | 1 |
| Spindle cell melanoma, NOS | Melanoma | UVM | 19 | TCGA | 1 |
| Spindle cell melanoma, type B | Melanoma | UVM | 9 | TCGA | 1 |
| Squamous cell carcinoma, keratinizing, NOS | Squamous cell carcinoma | CESC | 30 | TCGA | 1 |
| Squamous cell carcinoma, keratinizing, NOS | Squamous cell carcinoma | ESCA | 5 | TCGA | 1 |
| Squamous cell carcinoma, keratinizing, NOS | Squamous cell carcinoma | HNSC | 57 | TCGA | 1 |
| Squamous cell carcinoma, keratinizing, NOS | Squamous cell carcinoma | LUSC | 13 | TCGA | 1 |
| Squamous cell carcinoma, large cell, | Squamous cell carcinoma | CESC | 51 | TCGA | 1 |
| nonkeratinizing, NOS | | | | | |
| Squamous cell carcinoma, large cell, | Squamous cell carcinoma | HNSC | 11 | TCGA | 1 |
| nonkeratinizing, NOS | | | | | |
| Squamous cell carcinoma, large cell, | Squamous cell carcinoma | LUSC | 3 | TCGA | 1 |
| nonkeratinizing, NOS | | | | | |
| Squamous cell carcinoma, NOS | NA | BECA | 1 | TCGA | 0 |
| Squamous cell carcinoma, NOS | Squamous cell carcinoma | CESC | 171 | TCGA | 1 |
| Squamous cell carcinoma, NOS | Squamous cell carcinoma | ESCA | 90 | TCGA | 1 |
| Squamous cell carcinoma, NOS | Squamous cell carcinoma | HNSC | 449 | TCGA | 1 |
| Squamous cell carcinoma, NOS | Squamous cell carcinoma | LUSC | 338 | TCGA | 1 |
| Squamous cell carcinoma, small cell, | Squamous cell carcinoma | LUSC | 1 | TCGA | 1 |
| nonkeratinizing | | | | | |
| Squamous cell carcinoma, spindle cell | Squamous cell carcinoma | HNSC | 1 | TCGA | 1 |
| Superficial spreading melanoma | Melanoma | SKCM | 2 | TCGA | 1 |
| Synovial sarcoma, biphasic | Sarcoma | SARC | 2 | TCGA | 1 |
| Synovial sarcoma, NOS | Sarcoma | SARC | 2 | TCGA | 1 |
| Synovial sarcoma, spindle cell | Sarcoma | SARC | 6 | TCGA | 1 |
| Teratocarcinoma | Testicular germ cell tumor | TGCT | 2 | TCGA | 1 |
| Teratoma, benign | Testicular germ cell tumor | TGCT | 5 | TCGA | 1 |
| Teratoma, malignant, NOS | Testicular germ cell tumor | TGCT | 3 | TCGA | 1 |
| Thymic carcinoma, NOS | Thymoma | THYM | 11 | TCGA | 1 |
| Thymoms, type A, malignant | Thymoma | THYM | 15 | TCGA | 1 |
| thymoma, type A, NOS | Thymoma | THYM | 2 | TCGA | 1 |
| Thymoma, type AB, malignant | Thymoma | THYM | 31 | TCGA | 1 |
| Thymoma, type AB, NOS | Thymoma | THYM | 7 | TCGA | 1 |
| Thymoma, type B1, malignant | Thymoma | THYM | 13 | TCGA | 1 |
| Thymoma, type B1, NOS | Thymoma | THYM | 1 | TCGA | 1 |
| Thymoma, type B2, malignant | Thymoma | THYM | 26 | TCGA | 1 |
| Thymoma, type B2, NOS | Thymoma | THYM | 5 | TCGA | 1 |
| Thymoma, type B3, malignant | Thymoma | THYM | 13 | TCGA | 1 |
| Transitional cell carcinoma | Adenocarcinoma | BLCA | 343 | TCGA | 1 |
| Tubular adenocarcinoma | Adenocarcinoma | BRCA | 1 | TCGA | 1 |
| Tubular adenocarcinoma | Adenocarcinoma | ESCA | 1 | TCGA | 1 |
| Tubular adenocarcinoma | Adenocarcinoma | READ | 5 | TCGA | 1 |
| Tubular adenocarcinoma | Adenocarcinoma | STAD | 75 | TCGA | 1 |
| Undifferentiated sarcoma | Sarcoma | SARC | 34 | TCGA | 1 |
| Yolk sac tumor | Testicular germ cell tumor | TGCT | 4 | TCGA | 1 |
| NA | NA | BRCA | 1 | TCGA | 0 |
| NA | NA | COAD | 2 | TCGA | 0 |
| NA | NA | GBM | 2 | TCGA | 0 |
| NA | NA | LGG | 1 | TCGA | 0 |
| NA | NA | READ | 1 | TCGA | 0 |
| NA | Testicular germ cell tumor | TGCT | 16 | TCGA | 1 |
| High grade serous ovarian cancer | Adenocarcinoma | OV | 99 | GSE133556 | 1 |
| Total | | | 8595 | | 7735 |

TABLE 1A
Baseline characteristics of the discovery data set.

| | | | Mean Age | Male N | Data |
|---|---|---|---|---|---|
| Cancer Type | Location | N | (SD) | (%) | Source |
| Adrenocortical | Adrenal gland | 80 | 47 (15.9) | 31 (38.8) | TCGA |
| adenocarcinoma | | | | | |
| Bladder adenocarcinoma | Bladder | 410 | 69 (10.6) | 303 (73.9) | TCGA |
| Breast adenocarcinoma | Breast | 761 | 59 (13.2) | 9 (1.2) | TCGA |
| Cervical adenocarcinoma | Cervix | 48 | 46 (12.3) | 0 (0) | TCGA |
| Cervical squamous cell | Cervix | 259 | 49 (14.0) | 0 (0) | TCGA |
| carcinoma | | | | | |
| Colorectal adenocarcinoma | Colon and rectum | 379 | 64 (12.9) | 202 (52.3) | TCGA |
| Glioma | Brain | 138 | 60 (12.8) | 80 (58.0) | TCGA |
| Kidney chromophobe | Kidney | 66 | 52 (14.3) | 39 (59.1) | TCGA |
| adenocarcinoma | | | | | |
| Kidney renal clear cell | Kidney | 319 | 61 (11.8) | 205 (64.3) | TCGA |
| adenocarcinoma | | | | | |
| Kidney renal papillary cell | Kidney | 275 | 62 (12.1) | 202 (73.5) | TCGA |
| adenocarcinoma | | | | | |
| Liver hepatocellular | Liver | 377 | 59 (13.5) | 255 (67.6) | TCGA |
| adenocarcinoma | | | | | |
| Lung adenocarcinoma | Lung | 458 | 65 (10.2) | 214 (46.7) | TCGA |
| Lung squamous cell carcinoma | Lung | 370 | 68 (8.7) | 274 (74.1) | TCGA |
| Pancreatic adenocarcinoma | Pancreas | 175 | 66 (11.0) | 98 (56.0) | TCGA |
| Prostate adenocarcinoma | Prostate | 498 | 61 (6.8) | 498 | TCGA |
| | | | | (100.0) | |
| Esophageal and head and | Esophagus and | 624 | 61 (11.7) | 467 (75.8) | TCGA |
| neck squamous cell carcinoma | head and neck | | | | |
| Cutaneous melanoma | Skin | 104 | 65 (13.9) | 62 (59.6) | TCGA |
| Uveal melanoma | Eye | 80 | 62 (14.0) | 45 (56.2) | TCGA |
| Esophageal and stomach | Esophagus and | 484 | 66 (10.9) | 336 (69.4) | TCGA |
| adenocarcinoma | stomach | | | | |
| Thyroid adenocarcinoma | Thyroid | 502 | 47 (15.8) | 133 (26.5) | TCGA |
| Mesothelioma | Pleura | 87 | 64 (9.8) | 71 (81.6) | TCGA |
| Pheochromocytoma and | Adrenal gland | 179 | 48 (15.1) | 78 (43.6) | TCGA |
| Paraganglioma | | | | | |
| Sarcoma | Soft tissues | 249 | 61 (14.7) | 113 (45.4) | TCGA |
| Testicular germ cell tumor | Testis | 150 | 32 (9.3) | 150 (100) | TCGA |
| Ovarian adenocarcinoma | Ovary | 109 | 49 (13.5)* | 0 (0) | TCGA, |
| | | | | | GSE133556 |
| Thymoma | Thymus | 124 | 59 (13.0) | 64 (51.6) | TCGA |
| Endometrial adenocarcinoma | Uterus | 430 | 65 (11.2) | 0 (0) | TCGA |
| Total | | 7735 | | | |

*Horvath methylation age inferred using the wateRmelon package in R for GSE133556

### B. Tumor Classifier Hierarchy and Tumor Type Specific CpG Identification

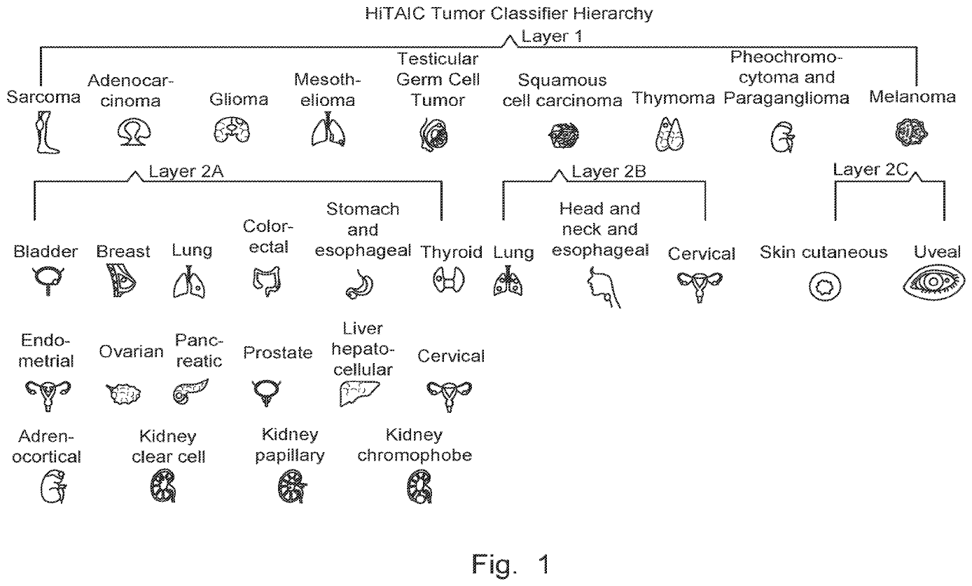

The tumor classifier hierarchy is established based on cancer pathophysiological differences and tissue location by tumor type. 2 layers with 4 categories can be established for 27 cancer types in the hierarchy (FIG. 1). Layer 1 contains 9 major tumor types. Layer 2A contains 16 types of adenocarcinoma. Layer 2B includes 3 types of squamous cell carcinoma. Layer 2C includes 2 types of melanoma. The sample labels are generated by examining the primary diagnosis and cancer-type information from TCGA following the HiTAIC hierarchy. Sample labels can be found on FigShare (DOI: 10.6084/m9.figshare.22179089). To reduce the high-dimensionality of the DNA methylation data with 384,640 CpGs, epigenome-wide association study (EWAS) is performed to identity differentially methylated CpGs across the cancer types in each category of the hierarchy as input for machine-learning model training using the whole discovery dataset to maximize the power. The Meffil (version 1.1.1) package in R (28) is applied, which uses limma linear regression with empirical Bayes adjustment statistics to reduce methylation profiles to top 100 cell-type-specific hyper- and hypomethylated CpGs per cancer type. Thus, 4 libraries of cancer-type discriminatory CpGs are developed. T-distributed stochastic neighbor embedding (T-SNE) is used to visualize the separation of cancers by methylation status from the Meffil selected CpGs in the libraries. R version 4.2.0 is used in this study.

### C. Machine Learning Model Development, Validation, and Application

By way of non-limiting example, all data mining and machine learning model building is operated in Jupyter Notebook Python 3 with the scikit-learn 1.1.1 package. See Fabian Pedregosa, G. V., Alexandre Gramfort, Vincent Michel, Bertrand Thirion, Olivier Grisel, Mathieu Blondel, Peter Prettenhofer, Ron Weiss, Vincent Dubourg, Jake Vanderplas, Alexandre Passos, David Cournapeau, Matthieu Brucher, Matthieu Perrot, Édouard Duchesnay. (2011) Scikit-learn: Machine Learning in Python. JMLR, 12(85):2825-2830. To select the best model, four different types of multi-class machine learning, models have been tested on the adenocarcinoma, squamous cell carcinoma, glioblastoma, and melanoma samples splitting to 80% training and 20% testing randomly. The support vector machine (SVM) model is established using the sklearn.svm.SVC function with gamma set as ‘auto’. The random forest classifier (RFC) are built using the sklearn.ensemble.RandomForestClassifier function with the number of trees set as 500. The Gaussian naïve Bayes (GNB) model is established using the sklearn.naive_bayes.GaussianNB function. Finally, the multilayer perceptron (MLP) model has been built using the sklearn.neural_network.MLPClassifier function with the max number of iterations set at 300. The performances of the models have been evaluated on the test data set using the sklearn.metrics.confusion matrix and sklearn.metrics.classification_report functions, which include stratified and overall precision, recall, and F1-score. Among four machine learning models applied, the MLP performs best in the test data set. Thus, MLP model is selected as the final model for cancer classification. In each layer of the hierarchy, an MLP model is trained using the selected cancer type discriminatory CpGs. In total, four MLPs are developed for the hierarchy. HiTAIC is then established based on the four hierarchical MLP models. Next, hyperparameter tuning is conducted to select the best set of hyperparameters for the MLP model in each layer of the hierarchy. A variety of techniques and numerical values can be employed in the tuning process, which should be clear to those of skill in the art. By way of non-limiting example of a hyperparameter tuning process, the hidden layer parameter is iterated through [100], [200], [500], [1000], [1000, 500], [1000, 200], [1000, 100], [500, 200], [500, 100], [200, 100], [1000, 500, 200], [1000, 500, 100], and [500, 200, 100]. The optimizer is iterated through “sgd” and “adam” optimizers. The learning rate is iterated through 0.0001, 0.0005, 0.001, 0.002, 0.005, and 0.01. To ensure that the MLP model is generalizable and the performance of the model is consistency across the discovery data set, a 5-fold cross-validation can be established, which splits the data into 80% training and 20% testing 5 times for model evaluation, in each layer of the hierarchy. To validate the results, 1175 samples have been identified with DNA methylation data on 24 cancer subtypes from 21 publicly available data sets on GEO and ArrayExpress (Table 1B below). See also Legendre, C. R., Demeure, M. J., Whitsett, T. G., Gooden, G. C., Bussey, K. J., Jung, S., Waibhav, T., Kim, S. and Salhia, B. (2016) Pathway Implications of Aberrant Global Methylation in Adrenocortical Cancer. PLoS One, 11, e0150629; Ramalho-Carvalho, J., Graca, I., Gomez, A., Oliveira, J., Henrique, R., Esteller, M. and Jeronimo, C. (2017) Downregulation of miR-130b~301b cluster is mediated by aberrant promoter methylation and impairs cellular senescence in prostate cancer. J Hematol Oncol, 10, 43; Oltra, S. S., Pena-Chilet, M., Vidal-Tomas, V., Flower, K., Martinez, M. T., Alonso, E., Burgues, O., Lluch, A., Flanagan, J. M. and Ribas, G. (2018) Methylation deregulation of miRNA promoters identifies miR124-2 as a survival biomarker in Breast Cancer in very young women. Sci Rep, 8, 14373; Baharudin, R., Ishak, M., Muhamad Yusof, A., Saidin, S., Syafruddin, S. E., Wan Mohamad Nazarie, W. F., Lee, L. H. and Ab Mutalib, N. S. (2022) Epigenome-Wide DNA Methylation Profiling in Colorectal Cancer and Normal Adjacent Colon Using Infinium Human Methylation 450K. Diagnostics (Basel), 12; Court, F., Le Boiteux, E., Fogli, A., Muller-Barthelemy, M., Vaurs-Barriere, C., Chautard, E., Pereira, B., Biau, J., Kemeny, J. L., Khalil, T. et al. (2019) Transcriptional alterations in glioma result primarily from DNA methylation-independent mechanisms. Genome Res, 29, 1605-1621; Worsham, M. J., Chen, K. M., Datta, I., Stephen, J. K., Chitale, D., Gothard, A. and Divine, G. (2016) The biological significance of methylome differences in human papilloma virus associated head and neck cancer. Oncol Lett, 12, 4949-4956; Ramalho-Carvalho, J., Goncalves, C. S., Graca, I., Bidarra, D., Pereira-Silva, E., Salta, S., Godinho, M. I., Gomez, A., Esteller, M., Costa, B. M. et al. (2018) A multiplatform approach identifies miR-152-3p as a common epigenetically regulated onco-suppressor in prostate cancer targeting TMEM97. Clin Epigenetics, 10, 40; Shen, J., Wang, S., Zhang, Y. J., Wu, H. C., Kibriya, M. G., Jasmine, F., Ahsan, H., Wu, D. P., Siegel, A. B., Remotti, H. et al. (2013) Exploring genome-wide DNA methylation profiles altered in hepatocellular carcinoma using Infinium HumanMethylation 450 BeadChips. Epigenetics, 8, 34-43; Mirhadi, S., Tam, S., Li, Q., Moghal, N., Pham, N. A., Tong, J., Golbourn, B. J., Krieger, J. R., Taylor, P., Li, M. et al. (2022) Integrative analysis of non-small cell lung cancer patient-derived xenografts identifies distinct proteotypes associated with patient outcomes. Nat Commun, 13, 1811; Yamamoto, Y., Matsusaka, K., Fukuyo, M., Rahmutulla, B., Matsue, H. and Kaneda, A. (2020) Higher methylation subtype of malignant melanoma and its correlation with thicker progression and worse prognosis. Cancer Med, 9, 7194-7204; Park, J. L., Jeon, S., Seo, E. H., Bae, D. H., Jeong, Y. M., Kim, Y., Bae, J. S., Kim, S. K., Jung, C. K. and Kim, Y. S. (2020) Comprehensive DNA Methylation Profiling Identifies Novel Diagnostic Biomarkers for Thyroid Cancer. Thyroid, 30, 192-203; and Trimarchi, M. P., Yan, P., Groden, J., Bundschuh, R. and Goodfellow, P. J. (2017) Identification of endometrial cancer methylation features using combined methylation analysis methods. PLoS One, 12, e0173242.

TABLE 1B
Baseline characteristics of the external validation data set.

| | | | Mean Age | | |
|---|---|---|---|---|---|
| Cancer Type | Location | N | (SD) | Male N (%) | Data Source |
| Adrenocortical adenocarcinoma | Adrenal gland | 18 | 37 (16.2)* | NA | GSE77871 |
| Bladder adenocarcinoma | Bladder | 25 | 58 (13.2)* | NA | GSE52955 |
| Breast adenocarcinoma | Breast | 188 | 60 (23.9)* | 0 (0) | GSE75067 |
| Cervical cancer | Cervix | 6 | 53 (21.8)* | 0 (0) | GSE169622 |
| Colorectal adenocarcinoma | Colon and rectum | 54 | 56 (15.5)* | 24 (44.4) | GSE193535 |
| Glioma | Brain | 70 | 84 (21.6)* | 51 (72.9) | GSE123678 |
| Head and neck squamous cell | Head and neck | 8 | 45 (15.5)* | 7 (87.5) | GSE67114 |
| carcinoma | | | | | |
| Kidney renal clear cell carcinoma | Kidney | 17 | 63 (12.4)* | NA | GSE52955 |
| Liver hepatocellular carcinoma | Liver | 66 | 65 (19.5)* | 50 (75.8) | GSE54503 |
| Lung adenocarcinoma | Lung | 47 | 66 (9.9) | 22 (46.8) | E-MTAB- |
| | | | | | 10156 |
| Lung squamous cell carcinoma | Lung | 57 | 67 (9.8) | 41 (71.9) | E-MTAB- |
| | | | | | 10156 |
| Pancreatic adenocarcinoma | Pancreas | 12 | 58 (8.7)* | NA | GSE74071 |
| Prostate adenocarcinoma | Prostate | 25 | 58 (6.7)* | 58 (100) | GSE52955 |
| Cutaneous melanoma | Skin | 46 | 69 (13.4) | 25 (54.3) | GSE140169 |
| Stomach adenocarcinoma | Stomach | 12 | 60 (20)* | NA | GSE164988 |
| Thyroid carcinoma | Thyroid | 27 | 54 (16.3)* | NA | GSE121377 |
| Uveal melanoma | Eye | 23 | 29 (12.3)* | 10 (43.4) | GSE156876 |
| Mesothelioma | Pleura | 79 | 71 (9.2) | 58 (73.4) | GSE164269 |
| Sarcoma | Soft tissue | 158 | 36 (21,7)* | NA | GSE167059 |
| Pheochromocytoma and | Adrenal gland | 22 | 50 (23.9)* | 8 (36.3) | GSE43293 |
| Paraganglioma | | | | | |
| Ovarian adenocarcinoma | Ovary | 65 | 42 (16.7)* | 0 (0) | GSE65820 |
| Testicular germ cell tumor | Testis | 130 | 19 (14.6)* | 130 (100) | GSE74104 |
| Thymoma | Thymus | 11 | 67 (10.1) | 11 (100) | GSE94769 |
| Endometrial carcinoma | Uterus | 9 | 69 (12.9) | 0 (0) | GSE93589 |
| Total | | 1175 | | | |

*Horvath methylation age inferred using wateRmelon

To further validate HiTAIC on samples with a low tumor purity, HiTAIC has been applied to TCGA adenocarcinoma samples with a tumor purity <30% based on HITIMED DNA methylation tumor cell deconvolution (See Zhang above, and the above-incorporated PCT Application). To demonstrate the application of the model in cancer metastasis, 175 samples with DNA methylation data have been identified on 5 cancer types with 6 different metastatic locations from 8 data sets on GEO and ArrayExpress (Table 1C below). See Timp, W., Bravo, H. C., McDonald, O. G., Goggins, M., Umbricht, C., Zeiger, M., Feinberg, A. P. and Irizarry, R. A. (2014) Large hypomethylated blocks as a universal defining epigenetic alteration in human solid tumors. Genome Med, 6, 61; Qu, X., Sandmann, T., Frierson, H., Jr., Fu, L., Fuentes, E., Walter, K., Okrah, K., Rumpel, C., Moskaluk, C., Lu, S. et al. (2016) Integrated genomic analysis of colorectal cancer progression reveals activation of EGFR through demethylation of the EREG promoter. Oncogene, 35, 6403-6415; Jurmeister, P., Scholer, A., Arnold, A., Klauschen, F., Lenze, D., Hummel, M., Schweizer, L., Blaker, H., Pfitzner, B. M., Mamlouk, S. et al. (2019) DNA methylation profiling reliably distinguishes pulmonary enteric adenocarcinoma from metastatic colorectal cancer. Mod Pathol, 32, 855-865; and Ylitalo, E. B., Thysell, E., Landfors, M., Brattsand, M., Jernberg, E., Crnalic, S., Widmark, A., Hultdin, M., Bergh, A., Degerman, S. et al. (2021). A novel DNA methylation signature is associated with androgen receptor activity and patient prognosis in bone metastatic prostate cancer. Clin Epigenetics, 13, 133.

TABLE 1C
Baseline characteristics of the metastasized tumor data set.

| Original | Metastasized | | Mean Age | Male N | |
|---|---|---|---|---|---|
| Cancer Type | Location | N | (SD) | (%) | Data Source |
| Colon | Liver | 15 | 47 (10.4)* | NA | GSE53051, |
| | | | | | GSE77954 |
| Colon | Lung | 14 | 54 (24.1)* | NA | GSE53051, |
| | | | | | GSE116699 |
| Lung | Brain | 20 | 54 (6.5) | 4 (20) | E-MTAB-8660 |
| Prostate | Bone | 70 | 55 (15.8)* | 70 (100) | GSE174613 |
| Prostate | Liver | 4 | 70 (8.9) | 4 (100) | GSE116338 |
| Prostate | Lymph node | 2 | 78 (4.2) | 2 (100) | GSE116338 |
| Breast | Lymph node | 44 | 54 (18.3) | 0 (0) | GSE58999 |
| Testis | Lung | 3 | 0 (0) | 3 (100) | GSE156512 |
| Testis | Lymph node | 3 | 0 (0) | 3 (100) | GSE156512 |
| Total | | 175 | | | |

*Horvath methylation age inferred using wateRmelon

Additionally, 266 cfDNA samples have been identified with DNA methylation data from cancer patients in 5 types of cancer on GEO for application (Table 1D below). See Moss, J., Magenheim, J., Neiman, D., Zemmour, H., Loyfer, N., Korach, A., Samet, Y., Maoz, M., Druid, H., Arner, P. et al. (2018) Comprehensive human cell-type methylation atlas reveals origins of circulating cell-free DNA in health and disease. Nat Commun, 9, 5068; Hlady, R. A., Zhao, X., Pan, X., Yang, J. D., Ahmed, F., Antwi, S. O., Giama, N. H., Patel, T., Roberts, L. R., Liu, C. et al. (2019) Genome-wide discovery and validation of diagnostic DNA methylation-based biomarkers for hepatocellular cancer detection in circulating cell free DNA. Theranostics, 9, 7239-7250; Gordevicius, J., Krisciunas, A., Groot, D. E., Yip, S. M., Susic, M., Kwan, A., Kustra, R., Joshua, A. M., Chi, K. N., Petronis, A. et al. (2018) Cell-Free DNA Modification Dynamics in Abiraterone Acetate-Treated Prostate Cancer Patients. Clin Cancer Res, 24, 3317-3324; Silva, R., Moran, B., Russell, N. M., Fahey, C., Vlajnic, T., Manecksha, R. P., Finn, S. P., Brennan, D. J., Gallagher, W. M. and Perry, A. S. (2020) Evaluating liquid biopsies for methylomic profiling of prostate cancer. Epigenetics, 15, 715-727; and Silva, R., Moran, B., Baird, A. M., O'Rourke, C. J., Finn, S. P., McDermott, R., Watson, W., Gallagher, W. M., Brennan, D. J. and Perry, A. S. (2021) Longitudinal analysis of individual cfDNA methylome patterns in metastatic prostate cancer. Clin Epigenetics, 13, 168. The model is applied to the external validation data sets computing stratified and overall precision, recall, and F1-score to evaluate the performance. Next, the model is applied to the application data sets and used stratified and overall precision, recall, and F1-score to evaluate model performance in metastasized cancers and cfDNA from cancer patients.

TABLE 1D
Baseline characteristics of the tumor cfDNA data set.

| | | Mean | Male | Data |
|---|---|---|---|---|
| Cancer Type | N | Age (SD) | N (%) | Source |
| Breast adenocarcinoma | 3 | 59 (14.9) | 0 (0) | GSE122126 |
| Colon adenocarcinoma | 4 | 71 (24.2) | 2 (50) | GSE122126 |
| Liver hepatocellular | 22 | 47 (15.4)* | NA | GSE129374 |
| adenocarcinoma | | | | |
| Lung small cell | 4 | 68 (12.4) | 2 (50) | GSE122126 |
| and non-small | | | | |
| cell carcinoma | | | | |
| Prostate | 233 | 65 (10.5)* | 233 (100) | GSE108462, |
| adenocarcinoma | | | | GSE119260, |
| | | | | GSE157273 |
| Total | 266 | | | |

*Horvath methylation age inferred using wateRmelon

### D. Functional Pathway and Genomic Context Enrichment Analyses

To explore the potential biological pathways and functions related to the CpGs distinguishing cancers, the Genomic Regions Enrichment of Annotations Tool (GREAT) is employed to perform enrichment analysis for cancer type specific CpGs in each layer in the hierarchy. GREAT uses Gene Ontology (GO) database, which includes biological process, cellular component, and molecular function categories. False discovery rate (FDR) is used to select and rank the significantly enriched GO terms (FDR<0.05). Next, genomic context enrichment analyses is conducted to investigate whether the cancer type specific CpGs are enriched in certain genomic locations. The relation of probes to CpG islands and enhancers are identified from the HumanMethylation450K annotation file. To define the genomic regions as promoters, introns, exons, or intergenic for each probe, the annotateWithGeneParts function from the R-package genomation and the UCSC_hg19_refGene file are used to map the regions to all CpG loci on the Illumina HumanMethylation450K array. If a probe mapped to more than a single genomic region, the probe is assigned preferentially with the order: promoters, exons, introns, and intergenic. Fisher's exact tests are conducted to calculate odds ratios (ORs), p-values, and 95% confidence intervals for genomic context enrichment analysis. For both functional pathway and genomic context enrichment analyses, cancer discerning CpGs in each layer are tested over the background CpGs used (n=384,640) for EWAS.

### E. HiTAIC Web-based Application and Computing Environment

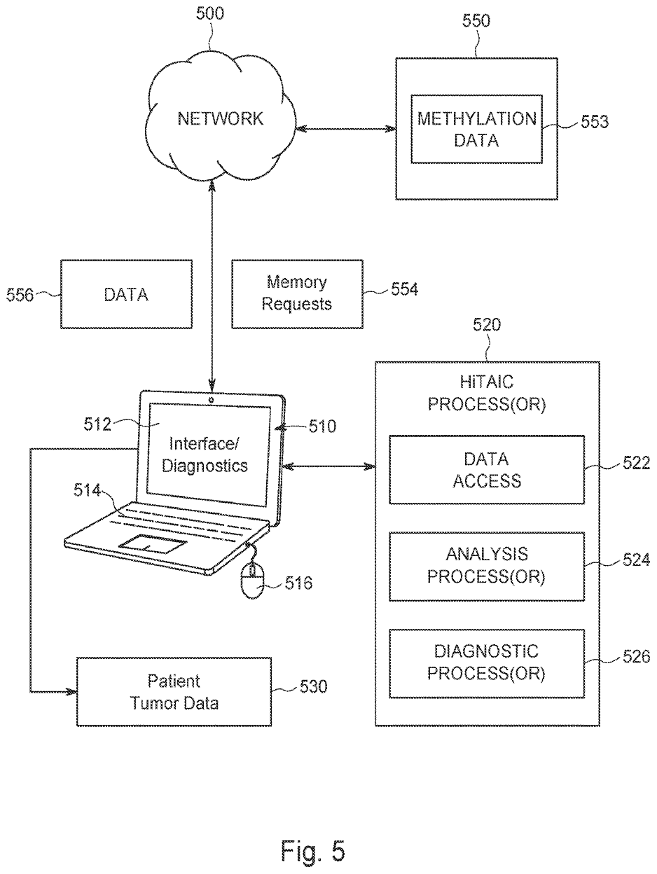

The computing environment and associated tool are shown in the computing and processing arrangement 500 of FIG. 5, which shows a generalized computing environment/system 500 for performing the tasks of the system and method herein. The system 500 includes at least one computing device 510 in the form of a general purpose computer (e.g., a PC, laptop, tablet, server, cloud computing arrangement, etc.) that includes an interface screen (e.g., touchscreen) 512, and various user interface devices (e.g. keyboard 514 and mouse 516). The computing device 510 instantiates a process(or) 520 that operates the data handling and diagnostic tasks herein. The computing device 510 receives patient tumor (and other related) data 530—via manual input, network based-inputs from patient records and/or from appropriate medical devices. The computing device 510 is further connected, via an appropriate wired and/or wireless link to a public and/or private data network (such as the Internet) 540 that allows access to one or more data stores 550 having relevant DNA methylation data 553 related to tumor types, etc. as described herein. Access consists of requests 554 for particular information, which result in the return of relevant data 556 for use in the process(or) 520. The date store 550 can be constructed using any appropriate data structure, including well-known database arrangements, and can be distributed among a plurality of data stores managed by one or multiple entities. Requests 554 are directed to the appropriate store based upon a known addressing scheme.

The process(or) 520 can be arranged in any acceptable configuration clear to those of skill, and the functional processes/ors or modules depicted are by way of non-limiting example. The process(or) 520 includes a data access process(or) 522 that handles patient data on tumors and user inputs to issue appropriate requests 554 to the data store 550 and retrieve relevant data 556. The data is used by the analysis process(or) 524 to perform a relevant processing using (e.g.) machine learning and trained classifiers to operate on presented data. This can be facilitated by appropriate comparison routines, including those supported by commercially available (or custom) Artificial Intelligence (AI) based systems, including, but not limited to Neural Networks, Convolutional Neural Networks (CNNs), and similarly functioning systems. The results of the analysis can be presented as a diagnosis with associated data on the condition by a diagnostic process(or) 526 using various stored and/or derived (via programmed algorithms/processes) that interoperate with results from the analysis process(or) 524.

By way of further background, Python Dash (See WorldWideWeb URL address https://dash.plotly.com/dash-html-components/cite) and Heroku (See WorldWideWeb URL address https://www.heroku.com/) are used to develop a user-friendly web-based HiTAIC tool. Python Dash is a framework developed by Plotly, which is based on Python, and used for building and deploying data applications with a customized interface. Heroku, which is (e.g.) a cloud platform, is next employed to host and deploy the HITAIC web application. The HiTAIC web application running in the process(or) 520 contains two major parts. The first part is a user guide. Users should follow the instructions to finish the prediction process. An exemplary input data csv file is available to the user for demonstration. The second part includes data upload, model running, and output download. After constructing the input data as instructed, users (operating the interface 512, 514 and 516) can either click the data upload box to choose the file or drag the file to the box from the local end to upload the input data. Then the algorithm will automatically compute the output and show up on the right side of the panel. Users can also download the output result as a csv file by clicking the export box.

In operation, the application can include appropriate APIs that allow it to interoperate with the operating system and/or web browser of the users computing device(s). Appropriate security functionality can be installed in the user's device (e.g. SSL-based communication frameworks) the ensure confidentiality of patient and user information. Availability of the software application (e.g. for download and/or installation) can be provided via an appropriate web-based distribution source that operates a server for such downloads (e.g. Google Play, Apple Store, etc.). The revenue model, if applicable, can be based on a subscription service and/or use of validated credentials that are established for each user and employed when logging into the application to enter, manipulate or view data. Such arrangements should be clear to those of skill in the art.

### III. Results of System and Method and Further Considerations

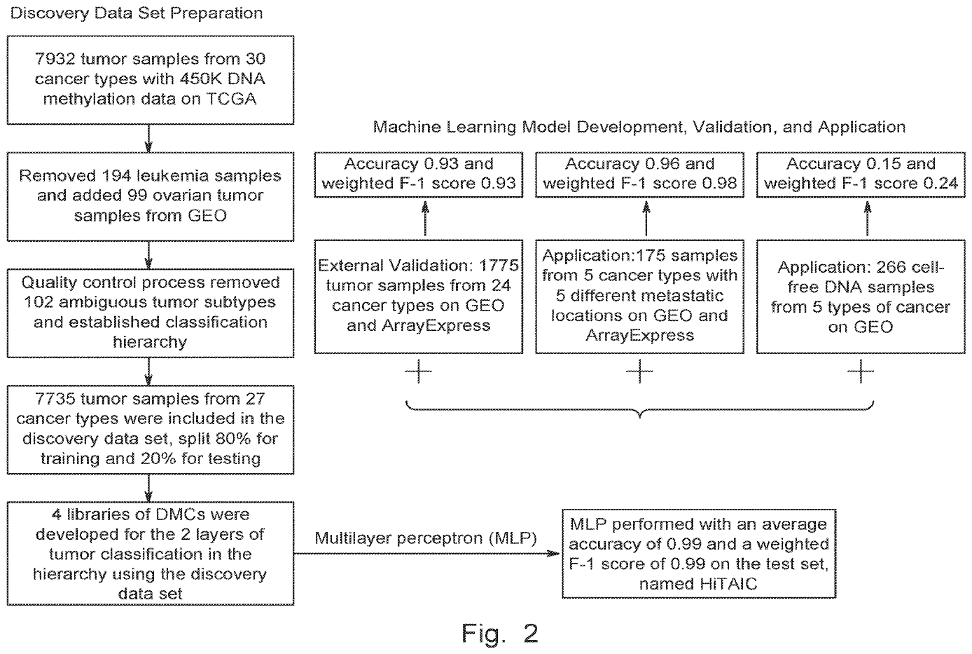

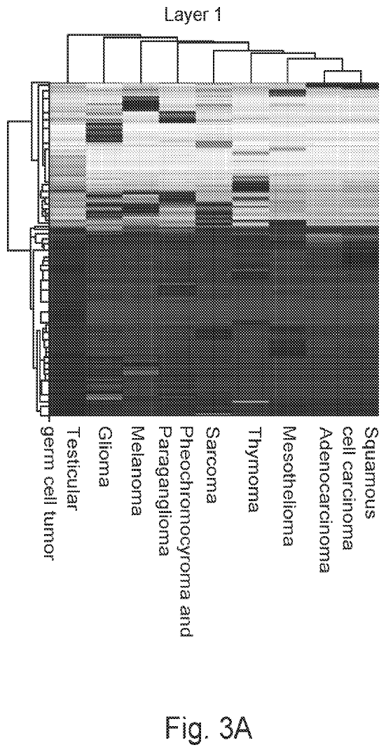

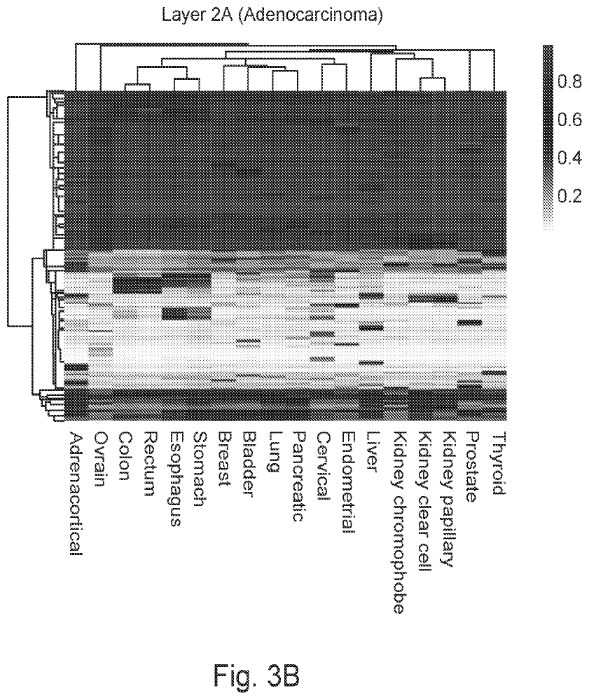

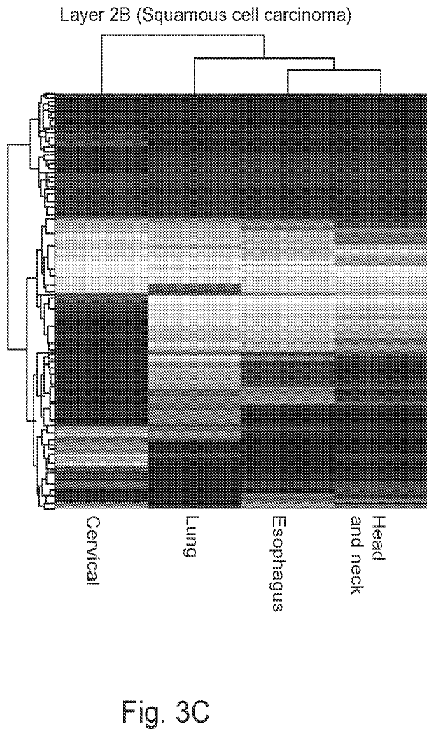

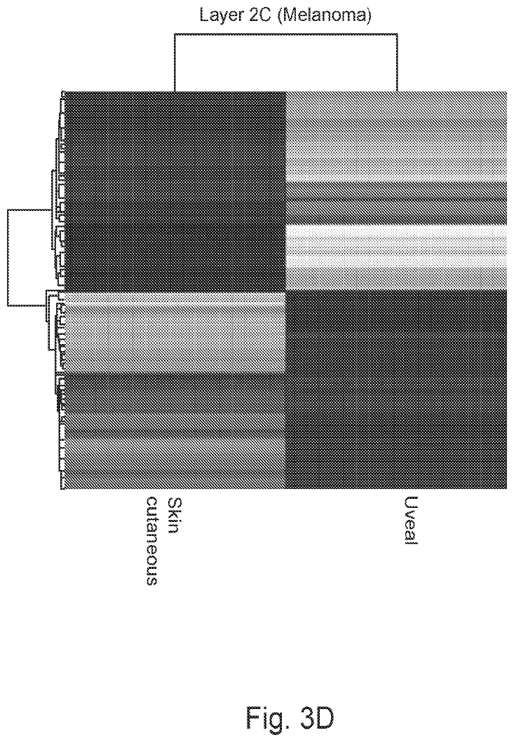

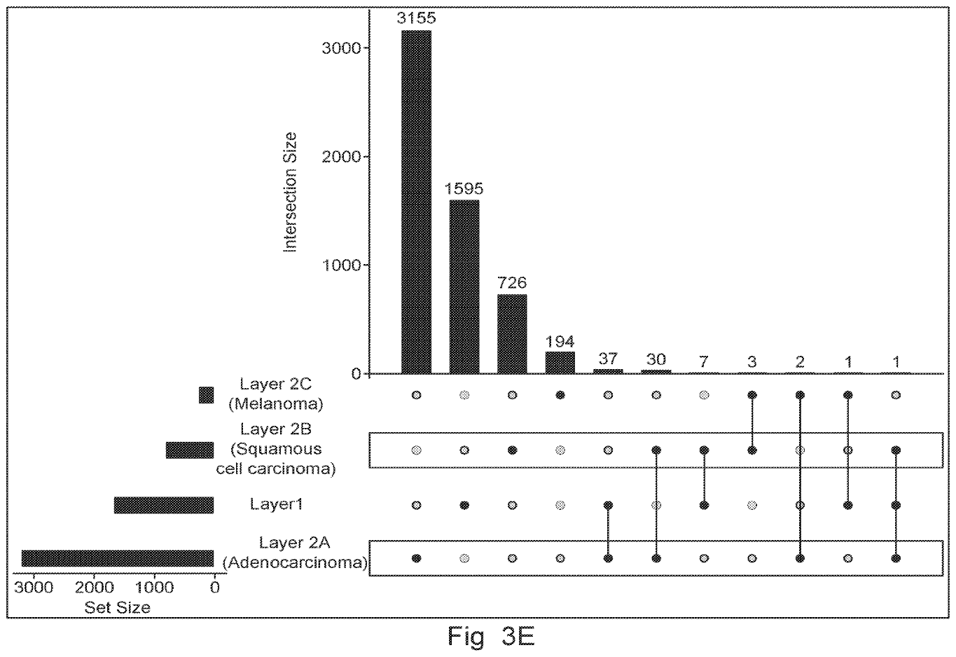

The pipeline of the operations presented herein is shown in FIG. 2. In total, four exemplary libraries of cancer type discriminatory CpGs are developed for the hierarchy. In Layer 1, 1641 CpGs are entified to discern 9 major cancer types. In Layer 2A, 3225 CpGs are identified to distinguish 18 adenocarcinoma cancer types. In Layer 2B, 767 CpGs are identified to discriminate 4 squamous cell carcinoma cancer types. In Layer 2C, 200 CpGs are identified to discern 2 types of melanoma. The heatmaps in FIGS. 3A-3D demonstrate discriminative methylation status for the cancer type specific CpGs in the libraries. The libraries are relatively unique to each other as 0 overlapped CpGs are identified across the 4 libraries, 1 CpG appeared in 3 out of 4 libraries, and 80 CpGs in total overlapped in 2 out of 4 libraries (FIG. 3E).

T-SNE clustering shows separation of clusters by cancer type using the cancer type discriminatory CpGs. However, certain cancer types do not show clear separation. Esophageal carcinoma is split into clusters with head and neck squamous cell carcinoma and stomach adenocarcinoma. Colon and rectum adenocarcinoma samples are basically indistinguishable in T-SNE. The two clusters of esophageal carcinoma can be separated by tumor subtypes, i.e. squamous cell carcinoma versus adenocarcinoma. Esophageal squamous cell carcinoma is clustered with head and neck squamous cell carcinoma, while esophageal adenocarcinoma is clustered with stomach adenocarcinoma. To avoid ambiguity and ensure the sensitivity of the model, colon adenocarcinoma and rectal adenocarcinoma are collapsed into colorectal adenocarcinoma in the adenocarcinoma layer, esophageal and head and neck squamous cell carcinoma into one group in the squamous cell carcinoma layer, and esophageal and stomach adenocarcinoma into one group in the adenocarcinoma layer in the hierarchy for MLP model training. Indicating differential methylation status by cancer types and feasibility for machine learning model training the T-SNE clustering showed clear separation of cancer types by using the cancer type discriminatory CpGs following the hierarchical cancer classification regime with minimal outliers.

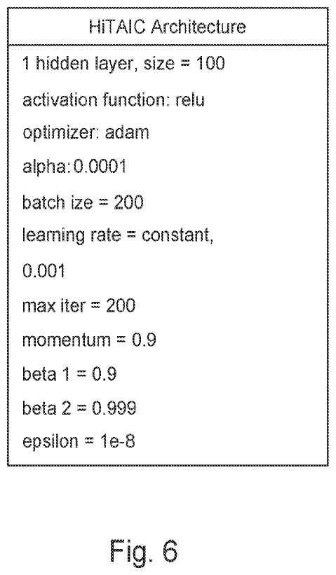

Next, HiTAIC can be trained using the MLP models and cancer type discriminatory CpGs using the training data set following the hierarchical structure. HiTAIC integrated four MLP models for tracing tumor tissue of origin. With the 156 sets of hyperparameters examined, the accuracy has ranged from 96%-98%, 82%-98%, 87%-99%, and 100% for Layer 1, Layer 2A, Layer 2B, and Layer 2C respectively. As a result, 1 hidden layer with 100 nodes, “adam” optimizer, and 0.001 learning rate are selected as the hyperparameters for the final model. The architecture of the HiTAIC model is shown in FIG. 6. Specifically, in the testing data set, high accuracy of HiTAIC's performance has been observed. In Layer 1, HiTAIC performs with 98% accuracy and 98% weighted average F1-score (Table 2 below). In Layer 2A with adenocarcinoma, HiTAIC performs with 98% accuracy and 98% weighted average F1-score (Table 3 below). In Layer 2B with squamous cell carcinoma, HiTAIC performs with 99% accuracy and 99% weighted average F1-score (Table 4 below). In Layer 2C with melanoma, HiTAIC performs with 100% accuracy and 100% weighted average F1-score (Table 5 below). For 5-fold cross-validation, the model performs consistently well in every layer of the hierarchy. In Layer 1, the accuracy and weighted average F1-score are all 98%. In Layer 2A, the accuracy and weighted average F1-score ranges from 98%. In Layer 2C, the accuracy and weighted average F1-score ranged from 97% to 100%. HiTIMED identified 101 TCGA adenocarcinoma samples with a tumor purity below 30%. See also Zhang and the above-incorporated PCT Application. HiTAIC achieves 97% accuracy and 98% weighted average F1-score. For external validation, a 93% accuracy and 93% weighted average F1-score across 25 cancer types is observed (Table 6 below). Specifically, all cancer types show a F1-score over 80% except for endometrial adenocarcinoma (F1-score 51%) and ovarian adenocarcinoma (F1-score 77%). Among 65 ovarian adenocarcinomas in the validation data set, 16 may be misclassified. All of these are (presently) misclassified as endometrial adenocarcinoma, resulting in a compromised F1-score for endometrial adenocarcinoma and ovarian adenocarcinoma. Although the F1-scores are (presently) relatively low in endometrial adenocarcinoma and ovarian adenocarcinoma, the misclassification is contained within the gynecologic cancer types, which still provides valuable information for tracing tumor tissue of origin.

TABLE 2
HiTAIC performance on the test data set for Layer 1 cancer types.

| | | | F1- | Sample |
|---|---|---|---|---|
| Layer 1 | Precision | Recall | score | Size |
| Adenocarcinoma | 0.99 | 0.99 | 0.99 | 1075 |
| Glioma | 1 | 1 | 1 | 27 |
| Melanoma | 0.97 | 0.97 | 0.97 | 37 |
| Mesothelioma | 1 | 0.94 | 0.97 | 17 |
| Pheochromocytoma and | 1 | 1 | 1 | 36 |
| Paraganglioma | | | | |
| Sarcoma | 0.92 | 0.96 | 0.94 | 50 |
| Squamous cell carcinoma | 0.97 | 0.94 | 0.96 | 251 |
| Testicular germ cell tumor | 1 | 1 | 1 | 30 |
| Thymoma | 1 | 1 | 1 | 25 |
| accuracy | | | 0.98 | 1548 |
| macro avg | 0.98 | 0.98 | 0.98 | 1548 |
| weighted avg | 0.98 | 0.98 | 0.98 | 1548 |

TABLE 3
HiTAIC performance on the test data set for Layer 2A
(adenocarcinoma) cancer subtypes.

| Layer 2A (Adenocarcinoma) | Precision | Recall | F1-score | Sample Size |
|---|---|---|---|---|
| Adrenocortical | 1 | 1 | 1 | 16 |
| Bladder | 0.96 | 1 | 0.98 | 82 |
| Breast | 0.99 | 1 | 1 | 152 |
| Cervical | 0.88 | 0.7 | 0.78 | 10 |
| Colorectal | 0.95 | 1 | 0.97 | 76 |
| Endometrial | 0.99 | 0.97 | 0.98 | 86 |
| Kidney chromophobe | 0.93 | 1 | 0.96 | 13 |
| Kidney clear cell | 0.98 | 0.92 | 0.95 | 64 |
| Kidney papillary cell | 0.96 | 0.95 | 0.95 | 55 |
| Liver hepatocellular | 1 | 1 | 1 | 75 |
| Esophageal and stomach | 1 | 1 | 1 | 97 |
| Lung | 0.99 | 0.98 | 0.98 | 92 |
| Ovarian | 0.96 | 1 | 0.98 | 22 |
| Pancreatic | 0.94 | 0.94 | 0.94 | 35 |
| Prostate | 1 | 1 | 1 | 100 |
| Thyroid | 1 | 1 | 1 | 100 |
| accuracy | | | 0.98 | 1075 |
| macro avg | 0.97 | 0.97 | 0.97 | 1075 |
| weighted avg | 0.98 | 0.98 | 0.98 | 1075 |

TABLE 4
HiTAIC performance on the test data set for Layer 2B
(squamous cell carcinoma) cancer subtypes.

| Layer 2B (Squamous | | | | Sample |
|---|---|---|---|---|
| cell carcinoma) | Precision | Recall | F1-score | size |
| Cervical | 1 | 0.98 | 0.99 | 52 |
| Esophageal and head and neck | 0.99 | 0.99 | 0.99 | 125 |
| Lung | 0.97 | 0.99 | 0.98 | 74 |
| accuracy | | | 0.99 | 251 |
| macro avg | 0.99 | 0.99 | 0.99 | 251 |
| weighted avg | 0.99 | 0.99 | 0.99 | 251 |

TABLE 5
HiTAIC performance on the test data set for
Layer 2C (melanoma) cancer subtypes.

| Layer 2C (Melanoma) | Precision | Recall | F1-score | Sample size |
|---|---|---|---|---|
| Eye uveal | 1 | 1 | 1 | 16 |
| Skin cutaneous | 1 | 1 | 1 | 21 |
| accuracy | | | 1 | 37 |
| macro avg | 1 | 1 | 1 | 37 |
| weighted avg | 1 | 1 | 1 | 37 |

TABLE 6
HiTAIC performance on the external validation data set.

| | | | F1- | Sample |
|---|---|---|---|---|
| Cancer Type | Precision | Recall | score | Size |
| Adrenocortical adenocarcinoma | 1 | 0.72 | 0.84 | 18 |
| Bladder adenocarcinoma | 0.78 | 1 | 0.88 | 25 |
| Breast adenocarcinoma | 0.96 | 1 | 0.98 | 188 |
| Cervical adenocarcinoma | 1 | 1 | 1 | 3 |
| Cervical squamous cell carcinoma | 1 | 0.67 | 0.8 | 3 |
| Colorectal adenocarcinoma | 1 | 0.89 | 0.94 | 54 |
| Endometrial adenocarcinoma | 0.35 | 1 | 0.51 | 9 |
| Eye uveal melanoma | 1 | 0.78 | 0.88 | 23 |
| Glioma | 0.97 | 1 | 0.99 | 70 |
| Esophageal and head and neck squamous cell carcinoma | 0.73 | 1 | 0.84 | 8 |
| Kidney clear cell carcinoma | 1 | 1 | 1 | 17 |
| Liver hepatocellular carcinoma | 0.99 | 1 | 0.99 | 66 |
| Esophageal and stomach adenocarcinoma | 0.92 | 1 | 0.96 | 12 |
| Lung adenocarcinoma | 0.96 | 0.98 | 0.97 | 47 |
| Lung squamous cell carcinoma | 0.98 | 0.86 | 0.92 | 57 |
| Mesothelioma | 1 | 0.85 | 0.92 | 79 |
| Ovarian adenocarcinoma | 0.79 | 0.75 | 0.77 | 65 |
| Pancreatic adenocarcinoma | 0.8 | 1 | 0.89 | 12 |
| Pheochromocytoma and Paraganglioma | 0.85 | 1 | 0.92 | 22 |
| Prostate adenocarcinoma | 0.89 | 1 | 0.94 | 25 |
| Sarcoma | 0.99 | 0.91 | 0.94 | 158 |
| Skin cutaneous melanoma | 0.9 | 1 | 0.95 | 46 |
| Testicular germ cell tumor | 0.94 | 0.93 | 0.93 | 130 |
| Thymoma | 1 | 0.82 | 0.9 | 11 |
| Thyroid adenocarcinoma | 1 | 1 | 1 | 27 |
| accuracy | | | 0.93 | 1175 |
| macro avg | 0.91 | 0.93 | 0.91 | 1175 |
| weighted avg | 0.95 | 0.93 | 0.93 | 1175 |

In metastasized cancer, HiTAIC demonstrated 96% accuracy and 9800 weighted average F1-score across 5 cancer types with 6 different metastatic locations (colon to liver, colon to lung, lung to brain, prostate to bone, prostate to liver, prostate to lymph node, breast to lymph node, testis to lung, testis to lymph node) (Table 7 below). In cfDNA from cancer patients, the model has low performance with 150% accuracy and a weighted F1-score of 24% (Table 8 below). Taken together, HiTAIC traces tumor tissue of origin and cancer subtype with a high accuracy in primary and metastasized cancer but not in cfDNA from cancer patients.

TABLE 7
HiTAIC performance on metastasized tumors.

| | | | | F1- | Sample |
|---|---|---|---|---|---|
| Original Cancer Type | Metastasized Location | Precision | Recall | score | Size |
| Breast | Lymph node | 1 | 0.98 | 0.99 | 44 |
| Colon | Liver, Lung | 1 | 0.93 | 0.96 | 29 |
| Lung | Brain | 0.94 | 0.85 | 0.89 | 20 |
| Prostate | Bone, Liver, Lymph node | 1 | 0.99 | 0.99 | 76 |
| Testicular germ cell tumor | Lung, Lymph node | 1 | 1 | 1 | 6 |
| accuracy | | | | 0.96 | 175 |
| macro avg | | 0.99 | 0.95 | 0.97 | 175 |
| weighted avg | | 0.99 | 0.96 | 0.98 | 175 |

TABLE 8
HiTAIC performance on cell-free DNA from cancer patients.

| | | | F1- | Sample |
|---|---|---|---|---|
| Cancer Type | Precision | Recall | score | Size |
| Breast Cancer | 0.33 | 1 | 0.5 | 3 |
| Colorectal Cancer | 1 | 0.75 | 0.86 | 4 |
| Liver Cancer | 1 | 0.09 | 0.17 | 22 |
| Lung Cancer | 0 | 0 | 0 | 4 |
| Prostate Cancer | 1 | 0.13 | 0.23 | 233 |
| accuracy | | | 0.15 | 266 |
| macro avg | 0.67 | 0.39 | 0.36 | 266 |
| weighted avg | 0.98 | 0.15 | 0.24 | 266 |

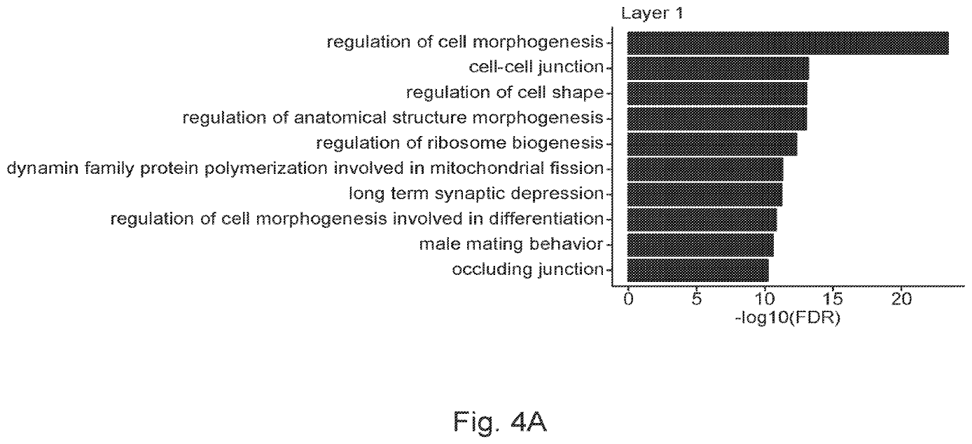

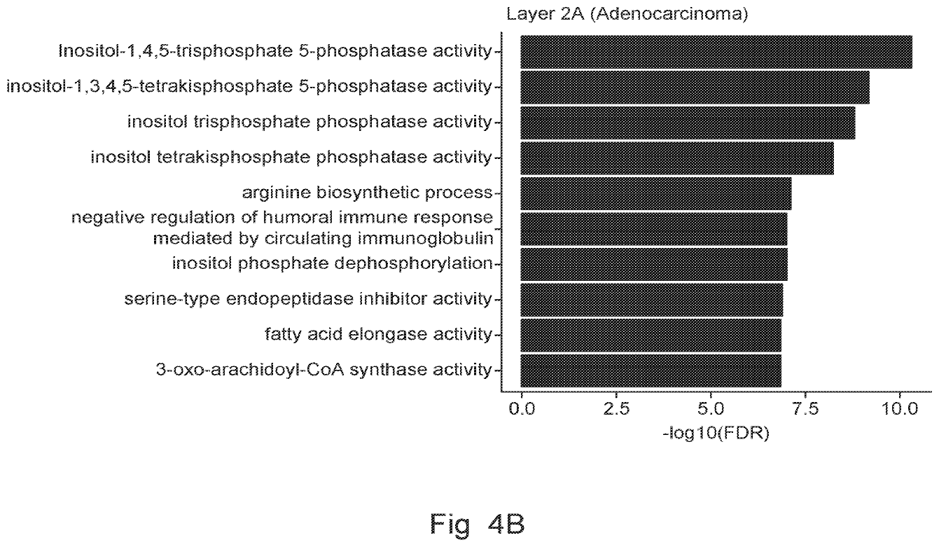

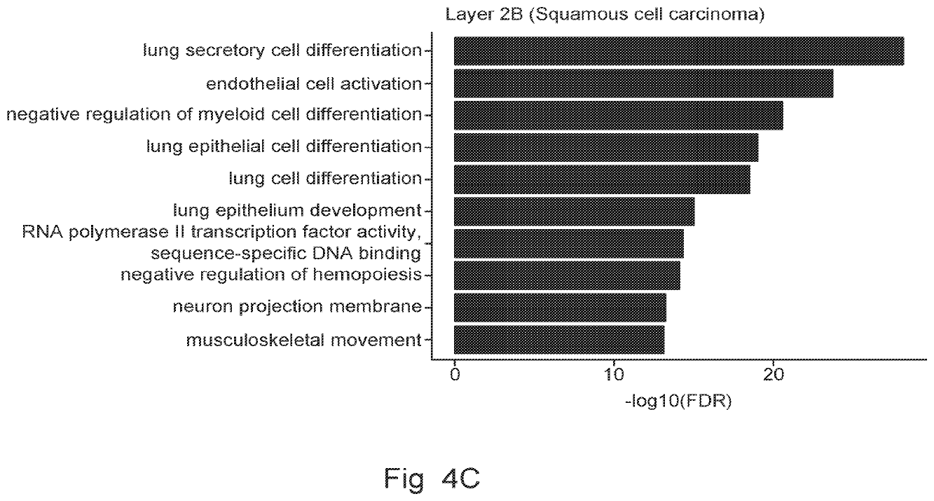

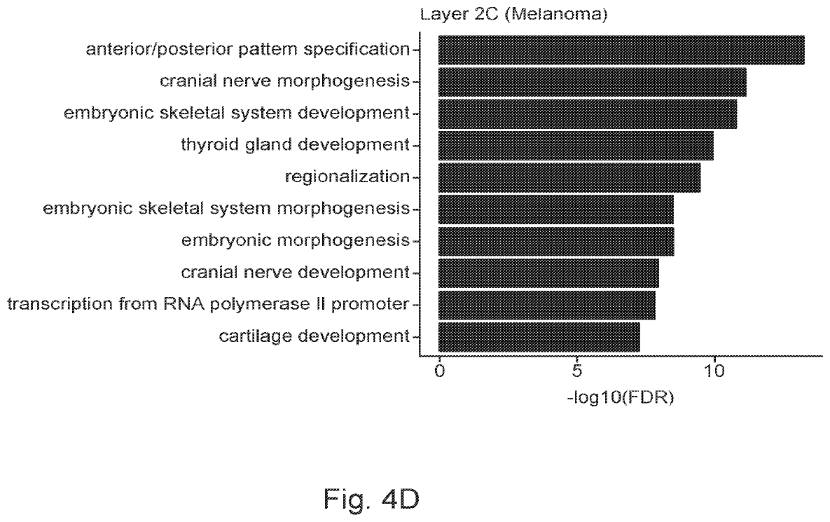

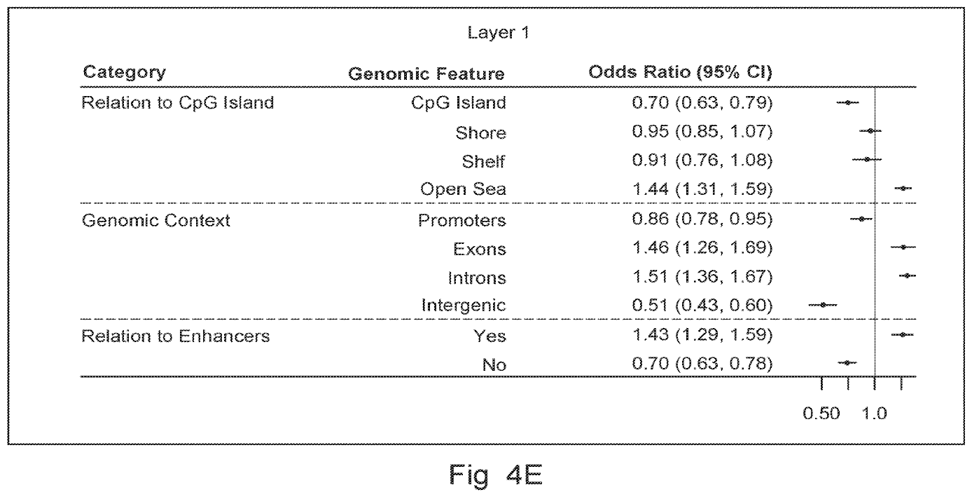

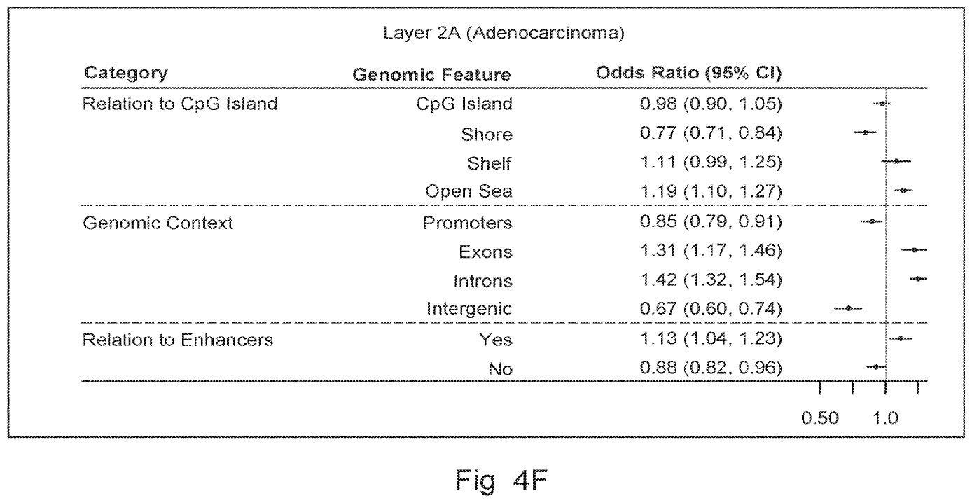

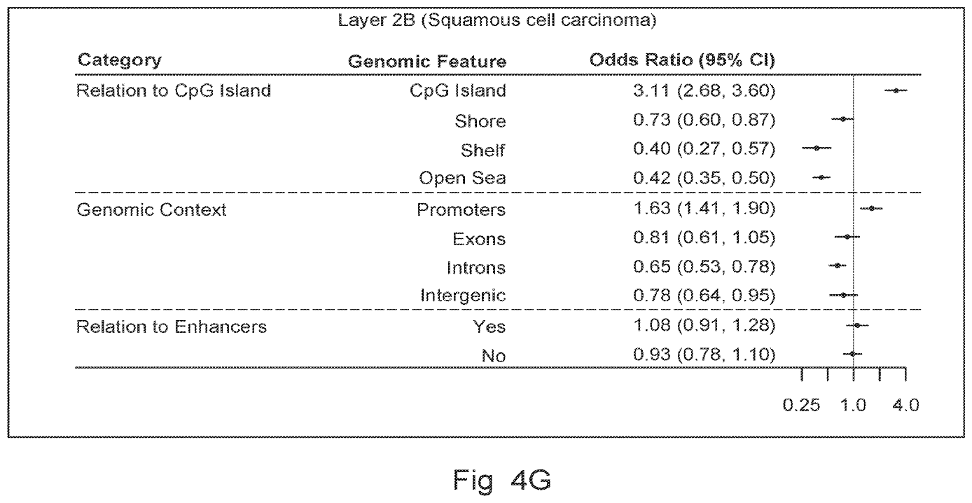

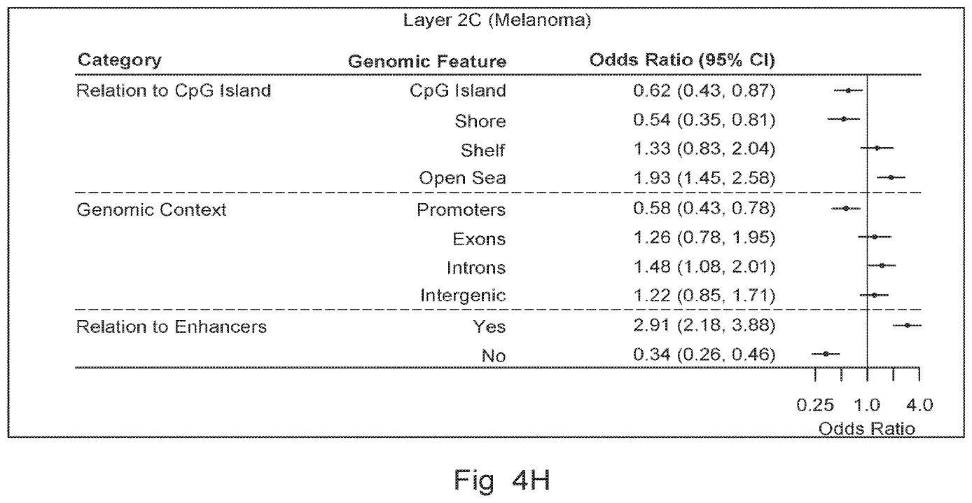

To investigate the biological pathways enriched for the cancer-discerning CpGs, a pathway enrichment analysis is conducted using GREAT. The investigation identified significantly enriched GO biological processes, cellular components, and molecular functions for each layer in the hierarchy. In Layer 1, the top 10 enriched biological pathways involve substantially cell differentiation and morphogenesis (FIG. 4A). In Layer 2A, which is designed for adenocarcinoma classification, the top 10 enriched biological pathways include majorly inositol phosphate metabolism (FIG. 4B). In Layer 2B, which is designed for squamous cell carcinoma classification, the top 10 enriched biological pathways contain mostly lung cell differentiation and development (FIG. 4C). In Layer 2C, which is designed for melanoma classification, the top 10 enriched biological pathways involve skeletal system development, regionalization process, and embryonic morphogenesis (FIG. 4D). Multiple genomic context enrichment analyses are conducted to investigate whether the cancer specific CpGs are enriched in certain genomic locations. The Layer 1 and Layer 2A CpGs are significantly enriched for open sea, exon, intron, and enhancer regions (FIG. 4E, FIG. 4F). The Layer 2B CpGs are significantly over-represented in CpG island and promoter regions (FIG. 4G). The Layer 2C CpGs are significantly enriched for open sea, intron, and enhancer regions (FIG. 4H).

DNA methylation is well-studied to show significant alteration in carcinogenesis, particularly hypermethylation in tumor suppressor genes and hypomethylation in oncogenes. See Ehrlich, M. (2009) DNA hypomethylation in cancer cells. Epigenomics, 1, 239-259. Although methylation alteration is a generic phenomenon in cancer, the across-cancer-type heterogeneity of genetic and epigenetic landscape enables distinguishable methylation patterns by tumor type. See Zhang, C., Zhao, H., Li, J., Liu, H., Wang, F., Wei, Y., Su, J., Zhang, D., Liu, T. and Zhang, Y. (2015). The identification of specific methylation patterns across different cancers. PLoS One, 10, e0120361. Furthermore, DNA methylation retains tissue and cell identities as it marks cell fate determination, which should enable identification of tumor tissue of origin and tumor type. Bogdanovic, O. and Lister, R. (2017) DNA methylation and the preservation of cell identity. Curr Opin Genet Dev, 46, 9-14. DNA methylation-based machine learning model, HiTAIC, is hereby developed to trace tissue of origin and tumor type in 27 primary tumor types. HiTAIC exhibits high performance in metastasized cancers.

Previous research has demonstrated the application of machine learning models on genetic and epigenetic data to develop clinical biomarkers for disease diagnosis and prognosis, including cancer. See Huang, S., Cai, N., Pacheco, P. P., Narrandes, S., Wang, Y. and Xu, W. (2018) Applications of Support Vector Machine (SVM) Learning in Cancer Genomics. Cancer Genomics Proteomics, 15, 41-51, and Sammut, S. J., Crispin-Ortuzar, M., Chin, S. F., Provenzano, E., Bardwell, H. A., Ma, W., Cope, W., Dariush, A., Dawson, S. J., Abraham, J. E. et al. (2022) Multi-omic machine learning predictor of breast cancer therapy response. Nature, 601, 623-629. DNA methylation-based machine learning models have demonstrated the capability to infer the location of unknown primary site from the site of metastasis. See Zheng, C. and Xu, R. (2020) Predicting cancer origins with a DNA methylation-based deep neural network model. PLoS One, 15, e0226461; Modhukur, V., Sharma, S., Mondal, M., Lawarde, A., Kask, K., Sharma, R. and Salumets, A. (2021) Machine Learning Approaches to Classify Primary and Metastatic Cancers Using Tissue of Origin-Based DNA Methylation Profiles. Cancers (Basel), 13; and Moran, S., Martinez-Cardus, A., Sayols, S., Musulen, E., Balana, C., Estival-Gonzalez, A., Moutinho, C., Heyn, H., Diaz-Lagares, A., de Moura, M. C. et al. (2016) Epigenetic profiling to classify cancer of unknown primary: a multicentre, retrospective analysis. Lancet Oncol, 17, 1386-1395. However, previous models have heavily favored inference of tissue site instead of tumor type, generating potential problems of indistinguishable tumor subtypes from the same site. Tumor cells originating from the same organ can exhibit widely varying histology and pathogenesis. For example, esophageal carcinoma can be dichotomized into two clusters by DNA methylation profile, one with head and neck squamous cell carcinoma and the other with stomach adenocarcinoma. Thus, a single-layer classification of “esophagus cancer” would conflate head and neck squamous cell carcinoma and stomach adenocarcinoma. To address the issue, a multilayer hierarchical approach is used, with differential DNA patterns by tumor type to achieve high-resolution tumor tissue of origin and subtype tracing. According to Zhang and the above-incorporated PCT Application, using DNA methylation data with hierarchical modeling achieves high-resolution deconvolution of the tumor microenvironment. In HiTAIC, adenocarcinoma and squamous cell carcinoma are initially distinguished, eliminating the potential issue of confusing esophageal adenocarcinoma with esophageal squamous cell carcinoma. Furthermore, previous work has limited cancer types and validation data sets, particularly with respect to metastasized cancers and cell-free DNA from cancer patients. Notably, previous work lacks a user-friendly and accessible tool for the generic scientific community. HiTAIC, conversely, includes 27 cancer types from 23 tissue sites in the discovery data sets, validated using external data sets, and demonstrated utility in external metastasized cancers. For easy accessibility of the process/algorithm and accommodate users without coding or other specialized computing knowledge/experience, a web-based application is developed and now available through the WorldWideWeb URL address https://sites.dartmouth.edu/salaslabhitaic/.

Significantly, HiTAIC traces tissue of origin and tumor type in metastasized cancers with a high accuracy, providing insight into the identification of CUP. Previous research has developed a methylation-based assay for tracing CUP, EPICUP, showed significantly better prognosis for CUP cases that are treated with site-specific therapy based on the assay compared to the CUP cases treated with empiric therapy, emphasizing the clinical significance of CUP diagnosis and directing the future of CUP diagnosis towards precision medicine. The hierarchical modeling approach employed by HiTAIC can maximize the power of detecting most differentially methylated CpGs in granular tumor subtypes and is a more research friendly bioinformatic tool that can serve the research community and facilitate research which involves inferring the primary tumor site of unknown origin.

Additionally, the predictability of the HiTAIC model is based on four libraries of CpGs discerning different layers of cancer subtypes. Interestingly, the four CpG libraries are almost completely distinct from each other with very low overlap. The biological pathways and genomic context enriched across the libraries are also diverse, indicating differential pathways involved in carcinogenesis and morphogenesis for different organ sites. In Layer 1, top enriched pathways are associated with cell differentiation, morphogenesis, and function. The distinguishable epigenetic regulation in cell differentiation may result from the substantially distinctive cancer types and tumor sites in Layer 1. In Layer 2A, which is designed for adenocarcinoma classification, inositol phosphate metabolism-related pathways are highly enriched. Inositol phosphate metabolic pathways are crucial for regulating cell migration, proliferation, apoptosis, and phosphatidylinositol-3-kinase (PI3K)/Akt signaling under normal physiological conditions. Studies have shown that dysregulation in inositol phosphate metabolism plays a key role in carcinogenesis. Gene variants in the inositol phosphate metabolism pathways are associated with risk of four types of cancer, including lung, esophageal, stomach, and kidney. See Tan, J., Yu, C. Y., Wang, Z. H., Chen, H. Y., Guan, J., Chen, Y. X. and Fang, J. Y. (2015) Genetic variants in the inositol phosphate metabolism pathway and risk of different types of cancer. Sci Rep, 5, 8473. Two studies have also shown that the association between inositol phosphate metabolism and cancer aggressiveness in both human and mouse models. See Benjamin, D. I., Louie, S. M., Mulvihill, M. M., Kohnz, R. A., Li, D. S., Chan, L. G., Sorrentino, A., Bandyopadhyay, S., Cozzo, A., Ohiri, A. et al. (2014) Inositol phosphate recycling regulates glycolytic and lipid metabolism that drives cancer aggressiveness. ACS Chem Biol, 9, 1340-1350, and Rao, F., Xu, J., Fu, C., Cha, J. Y., Gadalla, M. M., Xu, R., Barrow, J. C. and Snyder, S. H. (2015) Inositol pyrophosphates promote tumor growth and metastasis by antagonizing liver kinase Bl. Proc Natl Acad Sci USA, 112, 1773-1778. The biological implications of distinguishability for adenocarcinoma by differential epigenetic patterns regulating inositol phosphate metabolic pathways is intriguing and promises further investigation. Top pathways enriched for squamous cell carcinoma classification in Layer 2B involve lung cell differentiation, emphasizing tumor originating site specification to discern squamous cell carcinoma. To distinguish between cutaneous and uveal melanoma, top pathways can be enriched for embryonic development.

### IV. Conclusion

It should be clear to those of skill that the above-described system and method for implementing and using HiTAIC, consisting of a DNA methylation-based multilayer perceptron classifier to trace tissue of origin and tumor type in primary and metastasized tumors affords an effective technique tracing and identifying cancerous conditions and/or tumors in patients. The capability of the model described herein in tracing tumor origin and subtype, with high resolution and accuracy, can enable identification of cancer of unknown origin, and allow for development of effective treatment strategies and plans plan, which help to achieve precision medicine. HiTAIC can be easily deployed in a web-based application, which transforms the computational sophistication to a more user-friendly tool for public. More particularly, this system and method addresses limitations in existing methods and enhances the accuracy, utility and accessibility of tumor tracing. To achieve this goal, the system and method provides a novel DNA methylation-based process/algorithm that employs a tumor-type-specific hierarchical model and broadens the number of solid tumor types that are traced. HiTAIC uses multilayer perceptron models in combination with the discriminatory CpGs specific to tumor type in each layer in the hierarchy, to trace tumor tissue of origin and subtypes in (e.g) 27 primary and metastasized cancers. HiTAIC's ability to trace tumor tissue of origin with high resolution promises valuable application to clinical CUP identification.

### V. Publically Available Data

All data sets referenced herein are publicly available on The Cancer Genome Atlas (TCGA), Gene Expression Omnibus (GEO), and ArrayExpress. The accession numbers are GSE133556, GSE77871, GSE75067, GSE169622, GSE193535, GSE123678, GSE67114, GSE54503, E-MTAB-10156, GSE74071, GSE52955, GSE140169, GSE164988, GSE121377, GSE156876, GSE164269, GSE167059, GSE43293, GSE65820, GSE74104, GSE94769, GSE93589, GSE53051, GSE77954, GSEl 16699, E-MTAB-8660, GSE174613, GSE116338, GSE58999, GSE156512, GSE122126, GSE129374, GSE108462, GSE119260, and GSE157273. HiTAIC is publicly accessible on a user-friendly web page https://sites.dartmouth.edu/salaslabhitaic/. Code and documents are shared on FigShare (DOI: 10.6084/m9.figshare.22179089). Subsequent to the filing date of the above-incorporated U.S. Provisional Application, HiTAIC is publicly accessible via a user-friendly WorldWideWeb URL address, https://sites.dartmouth.edu/salaslabhitaic/.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments of the apparatus and method of the present invention, what has been described herein is merely illustrative of the application of the principles of the present invention. For example, as used herein, the terms “process” and/or “processor” should be taken broadly to include a variety of electronic hardware and/or software-based functions and components (and can alternatively be termed functional “modules” or “elements”). Moreover, a depicted process or processor can be combined with other processes and/or processors or divided into various sub-processes or processors. Such sub-processes and/or sub-processors can be variously combined according to embodiments herein. Likewise, it is expressly contemplated that any function, process and/or processor herein can be implemented using electronic hardware, software consisting of a non-transitory computer-readable medium of program instructions, or a combination of hardware and software. Additionally, as used herein various directional and dispositional terms such as “vertical”, “horizontal”, “up”, “down”, “bottom”, “top”, “side”, “front”, “rear”, “left”, “right”, and the like, are used only as relative conventions and not as absolute directions/dispositions with respect to a fixed coordinate space, such as the acting direction of gravity. Additionally, where the term “substantially” or “approximately” is employed with respect to a given measurement, value, or characteristic, it refers to a quantity that is within a normal operating range to achieve desired results, but that includes some variability due to inherent inaccuracy and error within the allowed tolerances of the system (e.g., 1-5 percent). Accordingly, this description is meant to be taken only by way of example, and not to otherwise limit the scope of this invention.

What is claimed is:

1. A system for tracing cancerous tissue in a patient using a processor and a user interface comprising:
a data input process that receives information related to the patient's cancerous tissue based on DNA methylation;
a data store that includes a plurality of layers of information arranged in a hierarchy relative to types of cancerous conditions in tissue based on DNA methylation therein;
an analysis process, including a trained machine learning process, that compares DNA methylation characteristics in the patient's tissue to the layers of information and performs a match so as to trace the cancerous conditions; and
a diagnostic process that provides a user with information relative to the match.

2. The system as set forth in claim 1, wherein a first layer of the plurality of layers is selected from a group of conditions consisting of: Adenocarcinoma, Glioma, Melanoma, Mesothelioma, Pheochromocytoma and Paraganglioma, Sarcoma, Squamous cell carcinoma, Testicular germ cell tumor, and Thymoma.

3. The system as set forth in claim 2, wherein the first layer relative to Adencarcinoma includes a second layer selected from a group of cancerous conditions consisting of those related to: Adrenocortical, Bladder, Breast, Cervical, Colorectal, Endometrial, Kidney chromophobe, Kidney clear cell, Kidney papillary cell, Liver hepatocellular, Esophageal and stomach, Lung, Ovarian, Pancreatic, Prostate, and Thyroid.

4. The system as set forth in claim 2, wherein the first layer relative to Squamous cell carcinoma includes a second layer selected from a group of cancerous conditions consisting of those related to: Cervical, Esophageal and head and neck, and Lung.

5. The system as set forth in claim 2, wherein the first layer relative to Melanoma includes a second layer selected from a group on cancerous conditions consisting of those related to: Eye uveal and Skin cutaneous.

6. The system as set forth in claim 1, wherein the analysis process is arranged to trace cancerous conditions from DNA methylation information in metastasized cancerous issue.

7. The system as set forth in claim 1, wherein the data store includes publicly available information accessed through a public data communication network.

8. The system as set forth in claim 1, wherein the machine learning process is trained using classifiers related to predetermined DNA methylation characteristics for related cancerous conditions.

9. The system as set forth in claim 1, wherein at least one of the data input process, the analysis process and the diagnostic process is operated using a processor on a user-controlled computing device with a user interface.

10. A method for tracing cancerous tissue in a patient using a processor and a user interface comprising the steps of:
receiving, at a processor arrangement, information related to the patient's cancerous tissue based on DNA methylation;
organizing, in a data store associated with the processor, a plurality of layers of information into a hierarchy relative to types of cancerous conditions in tissue based on DNA methylation therein;
analyzing, with a trained machine learning process, and comparing DNA methylation characteristics in the patient's tissue to the layers of information and performs a match so as to trace the cancerous conditions; and
providing to a user, with a diagnostic process, information relative to the match.

11. The method as set forth in claim 10, further comprising, selecting a first layer of the plurality of layers from a group of conditions consisting of: Adenocarcinoma, Glioma, Melanoma, Mesothelioma, Pheochromocytoma and Paraganglioma, Sarcoma, Squamous cell carcinoma, Testicular germ cell tumor, and Thymoma.

12. The method as set forth in claim 12, wherein the step of selecting the first layer relative to Adencarcinoma includes selecting a second layer from a group of cancerous conditions consisting of those related to: Adrenocortical, Bladder, Breast, Cervical, Colorectal, Endometrial, Kidney chromophobe, Kidney clear cell, Kidney papillary cell, Liver hepatocellular, Esophageal and stomach, Lung, Ovarian, Pancreatic, Prostate, and Thyroid.

13. The method as set forth in claim 11, wherein the step of selecting the first layer relative to Squamous cell carcinoma includes selecting a second layer from a group of cancerous conditions consisting of those related to: Cervical, Esophageal and head and neck, and Lung.

14. The method as set forth in claim 11, wherein the step of selecting the first layer relative to Melanoma includes selecting a second layer from a group on cancerous conditions consisting of those related to: Eye uveal and Skin cutaneous.

15. The method as set forth in claim 10, wherein the step of analyzing includes tracing cancerous conditions from DNA methylation information in metastasized cancerous tissue.

16. The method as set forth in claim 10, further comprising, building, in the data store, publicly available information accessed through a public data communication network.

17. The method as set forth in claim 10, further comprising, training the machine learning process using classifiers related to predetermined DNA methylation characteristics for related cancerous conditions.

18. The method as set forth in claim 10, further comprising, operating wherein at least one of the data input process, the analysis process and the diagnostic process is operated using a processor on a user-controlled computing device with a user interface.
